SEQUENCE LISTING (83 sequences)

SEQ ID NO: 1 - DNA, 1328 bp, Staphylococcus femA Consensus Sequence
  misc_feature (1)...(1328): n=any nucleotide
nnnnnnnnnnnnnanaatgaantttacnaatttnacngcnanaganttnngnnnntntacngannnnatgncnnanagncatttnacncanannnnngnnnantanganntnaannttgcnnannnnnnnganncncanntagtnggnatnaanaanaannataangangtnattgcngcntgnntnntnacngcngtnccngtnatgaaantnttnaantanttttattcnaanngnggnccngtnatngattntnanaannnaganctngtncantnnttctttaanganttnnnnaantatntnaaanannannntnnnntatanntnnnnntnganccntanntnncntatcaatannnnaatcatganggnganntnnnngnnaatgcnggnnangattggntnttngatnanntnnnnnnnntnggntntnancannnnggnttnnnnannggntttganccnntnnnncaaatnngntnncantcngtnntanatttannnnnnaaaannncnnanganntnntnaannnnatggatngnntnngnaanngnaanacnaaaaaagtnnanaanaatggngtnaaagtnnnnttnntnnnnnaaganganntnccnatnttnngntcattnatggangatacnncnganncnaangnnttnnnngatngngangannnnttntantanaanngntnnnnnnattnnaaagannnngtnntngtnccnntngcntatatnnantttgatgantanntnnnngaantnnannnnganngnnannnnntnantaaagannnnaanaaagcnntnaanganatnganaaangnccnganaanaaaaangcnnnnaanaannnnnnnaanntnnaanancaantnnnngcnaannancaaaanntnnangangnnannnnnntnnaannnnancatggnaangaattaccnatntcngcngnntncttnntnatnaatccntntgaagtngtntantangcnggtggnacntcnaatnnntnnngncanttngcnggnagntatgcnntncaatggnnnatgattaantatgcnntnnancatnnnatnnanngntanaatttntatggnnttagnggtnantttanngangangcngaagatgnnggngtnntnaanttnaaaaanggntnnnatgcnganntnntngantangttggnganttnntnaaaccnatnaanaanccnntntannnnnnntatannncantnaaaaanntnnannnnannnnnnnntannnannnnnnnannnnannnnnnnnnnnatgaaatttacagagttaannn

SEQ ID NO: 2 - DNA, 35 bp, Artificial Sequence
  Primer
anaatgaantttacnaatttnacngcnanagantt

SEQ ID NO: 3 - DNA, 20 bp, Artificial Sequence
  Primer
taatgaagtttacaaaattt

SEQ ID NO: 4 - DNA, 20 bp, Artificial Sequence
  Primer
taatgaagtttacnaaattt

SEQ ID NO: 5 - DNA, 25 bp, Artificial Sequence
  Primer
atgncnnanagncatttnacncana

SEQ ID NO: 6 - DNA, 20 bp, Artificial Sequence
  Primer
tgccatatagtcatttacgc

SEQ ID NO: 7 - DNA, 37 bp, Artificial Sequence
  Primer
tagtnggnatnaanaanaannataangangtnattgc

SEQ ID NO: 8 - DNA, 35 bp, Artificial Sequence
  Primer
gtnccngtnatgaaantnttnaantanttttattc

SEQ ID NO: 9 - DNA, 18 bp, Artificial Sequence
  Primer
aatgcnggnnangattgg

SEQ ID NO: 10 - DNA, 43 bp, Artificial Sequence
  Primer
gnaanngnaanacnaaaaaagtnnanaanaatggngtnaaagt

SEQ ID NO: 11 - DNA, 18 bp, Artificial Sequence
  Primer
aaaaagttcaaaaaatgg

SEQ ID NO: 12 - DNA, 18 bp, Artificial Sequence
  Primer
aaaaagtacaaaaaatgg

SEQ ID NO: 13 - DNA, 40 bp, Artificial Sequence
  Primer
aaganganntnccnatnttnngntcattnatggangatac

SEQ ID NO: 14 - DNA, 20 bp, Artificial Sequence
  Primer
tatatnnantttgatganta

SEQ ID NO: 15 - DNA, 32 bp, Artificial Sequence
  Primer
aanganatnganaaangnccnganaanaaaaa

SEQ ID NO: 16 - DNA, 18 bp, Artificial Sequence
  Primer
aaagatattgaaaaacga

SEQ ID NO: 17 - DNA, 20 bp, Artificial Sequence
  Primer
aaagatattgaaaagagacc

SEQ ID NO: 18 - DNA, 18 bp, Artificial Sequence
  Primer
aaagatatcgagaaagac

SEQ ID NO: 19 - DNA, 18 bp, Artificial Sequence
  Primer
aaagacatcgacaagcgt

SEQ ID NO: 20 - DNA, 22 bp, Artificial Sequence
  Primer
ancatggnaangaattaccnat

SEQ ID NO: 21 - DNA, 19 bp, Artificial Sequence
  Primer
gaacatggtaatgaattac

SEQ ID NO: 22 - DNA, 32 bp, Artificial Sequence
  Primer
aatccntntgaagtngtntantangcnggtgg

SEQ ID NO: 23 - DNA, 35 bp, Artificial Sequence
  Primer
agntatgcnntncaatggnnnatgattaantatgc

SEQ ID NO: 24 - DNA, 44 bp, Artificial Sequence
  Primer
tttanngangangcngaagatgnnggngtnntnaanttnaaaaa

SEQ ID NO: 25 - DNA, 20 bp, Artificial Sequence
  Primer
tttactgaagatgctgaaga

SEQ ID NO: 26 - DNA, 20 bp, Artificial Sequence
  Primer
gttggnganttnntnaaacc

SEQ ID NO: 27 - DNA, 20 bp, Artificial Sequence
  Primer
gttggtgactttattaaacc

SEQ ID NO: 28 - DNA, 19 bp, Artificial Sequence
  Primer
atgaaatttacagagttaa

SEQ ID NO: 29 - DNA, 18 bp, Artificial Sequence
  Primer
acagcagatgacatcatt

SEQ ID NO: 30 - DNA, 20 bp, Artificial Sequence
  Primer
taatgaaagaaatgtgctta

SEQ ID NO: 31 - DNA, 19 bp, Artificial Sequence
  Primer
acacaacttcaattagaac

SEQ ID NO: 32 - DNA, 17 bp, Artificial Sequence
  Primer
agtattagcaaatgcgg

SEQ ID NO: 33 - DNA, 17 bp, Artificial Sequence
  Primer
atgcatattttccgtaa

SEQ ID NO: 34 - DNA, 17 bp, Artificial Sequence
  Primer
cagcagatgacatcatt

SEQ ID NO: 35 - DNA, 23 bp, Artificial Sequence
  Primer
catctaaagatatattaaatgga

SEQ ID NO: 36 - DNA, 22 bp, Artificial Sequence
  Primer
agtattagcaaatgcgggtcac

SEQ ID NO: 37 - DNA, 20 bp, Artificial Sequence
  Primer
caacacaacttcaattagaa

SEQ ID NO: 38 - DNA, 20 bp, Artificial Sequence
  Primer
tggctatcgtgtcacaatcg

SEQ ID NO: 39 - DNA, 20 bp, Artificial Sequence
  Primer
ctggaacttgttgagcagag

SEQ ID NO: 40 - DNA, 1305 bp, Staphylococcus haemolyticus femA
  CDS (4)...(1266)
ataatgaagtttacaaatttaacagctacagagtttggcaattatacagataagatgccatatagtcatttcacacaaatgactgaaaactatgagatgaaagttgcaaataaaacagaaactcacttagttggtataaaaaataaagataatgaggttattgcagcctgcatgttgacagcagtaccagtcatgaaattttttaagtacttttattctaaccgaggacctgtaattgattatgataatagagagcttgttcactttttctttaatgagttaacaaagtatttaaaacagcataattgtctatatgttcgagttgacccttatttaccatatcaatatttaaatcatgatggtgaaattacaggtaatgctggtaatgattggttctttgataagatgaagcatctcggatttgaacatgaaggctttactaaaggttttgatccgattaaacaaatccgatatcattctgttttagatttaaaaaataaaacatctaaagatatattaaatggaatggatagtctacgtaaacgtaatactaaaaaagttcaaaaaaatggtgtgaaagttaagttcttatcagaagaagaacttccaatcttccgttcatttatggaagatacaaccgaaacgaaagaattccaagatagagatgatagtttctattataatcgctatagacatttcaaagatcacgtgcttgtaccactagcttatattaagtttgatgagtacatcgaagaattacaaaatgaacgtgaaactttaaataaagatgttaataaagctttaaaagatattgaaaaacgaccagacaataaaaaggcatttaataaaaaagaaaatcttgaaaaacaattagatgccaatcaacaaaaattagacgaggctaaaaaattacaagccgaacatggtaatgaattaccaatttcagcaggtttcttctttattaatccatttgaagttgtttattatgcaggtggaacttctaataaatatagacattttgcaggcagttatgctattcaatggacaatgattaactatgcaattgatcatggtattgatagatacaatttctatggtattagcggtaattttagtgaagacgctgaagatgttggagtcattaaatttaaaaaaggtttcaatgcagacgtaattgagtatgttggagactttgtgaaacctattaacaaacctttgtattcagtgtataagacactcaaaaagattaaaaaaagatttaattaaagaggggaatagacgaatatgaaatttacagagttaaac

SEQ ID NO: 41 - protein, 420 aa, Staphylococcus haemolyticus femA
MKFTNLTATEFGNYTDKMPYSHFTQMTENYEMKVANKTETHLVGIKNKDNEVIAACMLTAVPVMKFFKYFYSNRGPVIDYDNRELVHFFFNELTKYLKQHNCLYVRVDPYLPYQYLNHDGEITGNAGNDWFFDKMKHLGFEHEGFTKGFDPIKQIRYHSVLDLKNKTSKDILNGMDSLRKRNTKKVQKNGVKVKFLSEEELPIFRSFMEDTTETKEFQDRDDSFYYNRYRHFKDHVLVPLAYIKFDEYIEELQNERETLNKDVNKALKDIEKRPDNKKAFNKKENLEKQLDANQQKLDEAKKLQAEHGNELPISAGFFFINPFEVVYYAGGTSNKYRHFAGSYAIQWTMINYAIDHGIDRYNFYGISGNFSEDAEDVGVIKFKKGFNADVIEYVGDFVKPINKPLYSVYKTLKKIKKRFN

SEQ ID NO: 42 - DNA, 1280 bp, Staphylococcus lugdunensis femA
  CDS (1)...(1242)
acagcaaatgaattcggtgatttcacagatcaaatgccatatagtcattttactcaaatgacaggtaactataatttaaaagttgccgaaaaaacagaaacacatttagttggtgttaaaaataataataacgaagtaattgcagcatgtttattgacagctgtaccagtcatgaagttttttaaatacttttacagcaatagaggcccagttatagattatgctaaccaagaacttgtacattttttctttaatgagctaactaaatatttaaaaaagtataactgtctctatgtccgcatagatccatacttaccttatcaatatagagaccatgacggtaatataacggcaaatgctggcaatgattggtttttcaataaaatggaacaactcggataccatcatgatggctttacaacaggatttgatccaatattacaaatcagattccattctattcttaatttaaaggataagacagctaaagatgttttaaataatatggatagtttacgtaaaagaaataccaaaaaaagttcaaaaaatggagtcaaagtaaagttccttactgaagaagaactacctatctttcgttcatttatggagcagacgtcagaatctaaagaattctctgatagagacgaccaattttattacaatcggtttaagtactataaagatagggtgcttgtgcctctagcatatttaaaatttgatgaatatatagaagaactaacgaatgaacgacaaactttagaaaaagatttaggcaaagcacttaaagacattgagaaacgaccagataacaaaaaagcttataataaacgagacaacctacaacaacaactcgatgccaatcaacaaaagttaaatgaggctaatcagttacaagcggaacacggtaatgagttacctatctctgccggtttctttattattaatccgtttgaagttgtatactacgctggaggtaccgctaataaatatcgtcattttgcaggtagttacgcggttcagtggactatgattaactatgctatcgaacacggcatagacagatataatttctacggcattagtggaaacttctcagatgatgctgaagacgcaggtgtcattcgctttaaaaaaggttatggtgcagaagtgattgaatacgttggtgattttgtaaaacctataaataaacctatgtataaactttattcagtgttaaaacgaattcaaaataagctatagaggagaatggattaattatgaaatttacagagtttaac

SEQ ID NO: 43 - protein, 413 aa, Staphylococcus lugdunensis femA
TANEFGDFTDQMPYSHFTQMTGNYNLKVAEKTETHLVGVKNNNNEVIAACLLTAVPVMKFFKYFYSNRGPVIDYANQELVHFFFNELTKYLKKYNCLYVRIDPYLPYQYRDHDGNITANAGNDWFFNKMEQLGYHHDGFTTGFDPILQIRFHSILNLKDKTAKDVLNNMDSLRKRNTKKSSKNGVKVKFLTEEELPIFRSFMEQTSESKEFSDRDDQFYYNRFKYYKDRVLVPLAYLKFDEYIEELTNERQTLEKDLGKALKDIEKRPDNKKAYNKRDNLQQQLDANQQKLNEANQLQAEHGNELPISAGFFIINPFEVVYYAGGTANKYRHFAGSYAVQWTMINYAIEHGIDRYNFYGISGNFSDDAEDAGVIRFKKGYGAEVIEYVGDFVKPINKPMYKLYSVLKRIQNKL

SEQ ID NO: 44 - DNA, 1295 bp, Staphylococcus xylosus femA
  CDS (1)...(1245)
acgcaaaagagtttgggtgcattttcagataaaatgccaaatagccatttcacgcaaatggtagggaattatgaattgaaaattgcagaaagtactgaaacacatttagtaggtataaaaaacaatgataatgaagtcattgcagcttgtttattaactgcagtaccagtaatgaaattctttaagtatttttatactaatagaggtccggttatagattttgaaaataaagaattagtgcattactttttcaatgaactatctaaatatgtgaaaaaacataatgcgctttatttaagagttgatccttatttagcatatcaataccgtaatcatgatggtgaggtattggaaaatgcaggacatgattggattttcgataaaatgaagcagcttggatataaacaccaaggatttttaactggtttcgattcaattattcaaattaggttccactctgtactggatttagtaggtaaaactgctaaagatgtactaaatggtatggatagtttacgtaaacgtaatactaaaaaagtacaaaaaaatggcgtgaaagtaaggttcttaagggaagatgagttgccaattttccgttcattcatggaagatacatctgaaactaaagactttgacgatagagacgatggcttttactacaatagattaaggtattataaagatcgcgtattagtacctctagcttatatggatttcaatgaatatattgaagaattgcaagctgaacgtgaggtgttaagcaaagatatcaataaagcagtaaaagatatcgagaaaagacctgaaaataaaaaagcatataataaaaaagataatctagagaaacaacttatagcgaatcaacaaaaaattgatgaagctaaaactctacaagagaagcatggtaacgaactaccaatctcagcagcatatttcatcattaacccttatgaagtagtgtattatgcgggtggaacgtcaaatgagtttagacattttgctggtagttatgccattcaatggaagatgattaactatgctattgaccataatattgatagatataatttttatggaattagtggtcattttacagaagatgcagaagatgccggtgtagttaaatttaaaaaaggatttaatgcggatgtagtggaatatgttggtgattttattaaaccaatcaataaaccaatgtacaaaatttatacgacattaaagaaaattaaagataaaaagaaataaacatttaatagaagggaactaagctagaatgaaatttacagagttaaacc

SEQ ID NO: 45 - protein, 414 aa, Staphylococcus xylosus femA
TQKSLGAFSDKMPNSHFTQMVGNYELKIAESTETHLVGIKNNDNEVIAACLLTAVPVMKFFKYFYTNRGPVIDFENKELVHYFFNELSKYVKKHNALYLRVDPYLAYQYRNHDGEVLENAGHDWIFDKMKQLGYKHQGFLTGFDSIIQIRFHSVLDLVGKTAKDVLNGMDSLRKRNTKKVQKNGVKVRFLREDELPIFRSFMEDTSETKDFDDRDDGFYYNRLRYYKDRVLVPLAYMDFNEYIEELQAEREVLSKDINKAVKDIEKRPENKKAYNKKDNLEKQLIANQQKIDEAKTLQEKHGNELPISAAYFIINPYEVVYYAGGTSNEFRHFAGSYAIQWKMINYAIDHNIDRYNFYGISGHFTEDAEDAGVVKFKKGFNADVVEYVGDFIKPINKPMYKIYTTLKKIKDKKK

SEQ ID NO: 46 - DNA, 1283 bp, Staphylococcus capitis femA
  CDS (1)...(1236)
acagctaaagaatttagtgactttactgatcaaatgccttatagccattttactcagatggaaggtaattatgaacttaaagttgctgaaggtacggattcacatctcgtaggaattaaaaataatgacaaccaagtgattgcagcatgtttattaactgctgtacctgtaatgaaaatttttaaatatttttactcaaatcgcgggccagtgattgattatgataataaagagcttgttcactttttctttaatgaattaagtaaatatgtaaaaaagcataattgtctttatctaagagttgacccttatcttccttatcaatacttaaatcatgacggtgaaattattggaaatgctggccatgattggtttttcaataagatggaagaattaggatttgaacatgaaggctttcataaaggcttccatcctatcttacaagtaagatatcattcagttttagatttaaaagataaaacggctaaagatgtactcaaaggaatggatagtttaagaaagcgtaatactaagaaagtacaaaaaaatggtgtcaaagtccgtttcctatccgaagatgaattacctatctttagatcatttatggaagatactacagaaacgaaagagttcgccgatagagatgatagtttctattataatcgattaaaatactttaaagatagagtattagtaccattagcatatgttgacttcgatgagtatattgaagaacttaataatgaaagagatgttcttaataaagatttaaataaggcgctcaaagatattgagaagagacctgataataagaaagcttataacaaaagagataatcttcaacaacaattagatgcaaatcaacaaaaaattgatgaagctaaaaacttacaacaagaacatggtaatgaattacctatttcagctggatatttcttcattaatccgtttgaagttgtttattacgcaggtggcacatcgaatcgttatcgtcactatgccggaagttatgcaattcaatggaaaatgataaactatgctttagaacatggaattaaccgttataatttttatggagttagtggggacttcagtgaagacgctgaagatgtaggagtaattaagttcaaaaaaggctataatgctgatgttattgaatatgtaggtgattttatcaagccaatcaataaacctatgtatgcaatctataacgcacttaaaaagttaaagaaatagatttttttaccaacccaattatctaattatgaaatttacagagttaa

SEQ ID NO: 47 - protein, 411 aa, Staphylococcus capitis femA
TAKEFSDFTDQMPYSHFTQMEGNYELKVAEGTDSHLVGIKNNDNQVIAACLLTAVPVMKIFKYFYSNRGPVIDYDNKELVHFFFNELSKYVKKHNCLYLRVDPYLPYQYLNHDGEIIGNAGHDWFFNKMEELGFEHEGFHKGFHPILQVRYHSVLDLKDKTAKDVLKGMDSLRKRNTKKVQKNGVKVRFLSEDELPIFRSFMEDTTETKEFADRDDSFYYNRLKYFKDRVLVPLAYVDFDEYIEELNNERDVLNKDLNKALKDIEKRPDNKKAYNKRDNLQQQLDANQQKIDEAKNLQQEHGNELPISAGYFFINPFEVVYYAGGTSNRYRHYAGSYAIQWKMINYALEHGINRYNFYGVSGDFSEDAEDVGVIKFKKGYNADVIEYVGDFIKPINKPMYAIYNALKKLKK

SEQ ID NO: 48 - DNA, 1297 bp, Staphylococcus schleiferi femA
  CDS (1)...(1248)
acgacggctgaatttggtgcgtttacagatcaaatgccatatagccatttcacgcaaatggtagggaactatgaattaaaggttgctgaaggtgttgaaacacatcttgtcggcattaaagataacaacaataacgtactagcagcatgtttactgacagcagtgccagtaatgaagttttttaaatatttttattcaaaccgcggaccagtcatggactacgaaaataaagagctcgttcatttcttttttaatgaactttcaaaatatgttaagaaatatcacgcattgtatttgagagtagacccttatttaccaatgttaaagcgaaaccatgatggtgaagtgattgaaagatacggcagtgactggttttttgataaaatggctgaattaaactttgaacatgaaggtttcacaactgggtttgatacaataaggcaaattcgttttcattctgtgctcgatgttgaaaataaaacatcaaaagacatcttaaatcaaatggataatttaaggaaaagaaatacgaaaaaagtacagaaaaatggtgtgaaagtccgctatctaaacgaagatgaattacatattttccgttcgtttatggaagatacatctgaaacaaaagattttgtagatagagatgacgatttttattatcatcgtatgaaatactataaagatcgtgtccgcgtaccactagcgtatattgattttaatgcatatttagcagagctcaacactgaagcgcaagactttaaaaaagaaattgcaaaagcagataaagacatcgacaagcgtcctgaaaatcagaaagccataaataaaaagaaaaatttagagcaacaactagaagcgaatcaagctaaaataaaagaagcagaaacattgcaacttaaacacggtgacacattaccgatttcggctggattctttattattaatccatttgaggttgtttattatgcaggcggcacagcaaacgaatttcgtcattttgctggaagctacgcagtgcaatgggaaatgattaattatgcgattgattatcaaattccaagatataacttttatggcattagtggtgatttttcagaagatgcagaagatgcaggtgtgataaaatttaaaaaaggctataatgcagaagtaatagaatatgtcggtgattttattaagcctataaacaaacctgcctatacagtctacttaaaattaaagcaattaaaagacaagataaaaagataagatatagcaaagagaaggggatttattggtatgaaatttacagagttaa

SEQ ID NO: 49 - protein, 415 aa, Staphylococcus schleiferi femA
TTAEFGAFTDQMPYSHFTQMVGNYELKVAEGVETHLVGIKDNNNNVLAACLLTAVPVMKFFKYFYSNRGPVMDYENKELVHFFFNELSKYVKKYHALYLRVDPYLPMLKRNHDGEVIERYGSDWFFDKMAELNFEHEGFTTGFDTIRQIRFHSVLDVENKTSKDILNQMDNLRKRNTKKVQKNGVKVRYLNEDELHIFRSFMEDTSETKDFVDRDDDFYYHRMKYYKDRVRVPLAYIDFNAYLAELNTEAQDFKKEIAKADKDIDKRPENQKAINKKKNLEQQLEANQAKIKEAETLQLKHGDTLPISAGFFIINPFEVVYYAGGTANEFRHFAGSYAVQWEMINYAIDYQIPRYNFYGISGDFSEDAEDAGVIKFKKGYNAEVIEYVGDFIKPINKPAYTVYLKLKQLKDKIKR

SEQ ID NO: 50 - DNA, 1284 bp, Staphylococcus sciuri femA
  CDS (1)...(1233)
acactggaatttgaagcttttacaaataaaatgccgtacgcgcattttacacaagcagtaggtaattatgaattaaaaacatctgaaggtacttcaacacatttagtaggggtcaaagataatcaaggtgaagtattagctgcgtgtctgttaacaagtgtaccagttatgaagaaatttaattacttttactcaaatagaggaccagtaatggattatgacaacaaagaacttgttgactttttctttaaagaaatcgtgagctatttaaaaagttataaaggattattctttagaatcgatccttacttgccatatcaactaagagatcatgatggcaatattaaaaaatcattcaaccgtgatggtttaattaaacaatttgaatcattaggttatgaacaccaaggcttcacaactggtttccacccaatacatcaaattagatggcattctgtacttgatttagaaagtatggacgaaaagacgctcatcaagaacatggacagtttaagaaaaagaaatactaaaaaagttcaaaaaaatggtgttaaagttcgttttctatctaaagatgaaatgccgatattccgtcaatttatggaagatactacagagaagaaagatttcaacgatcgtggcgatgacttctattacaatagattaaaatactttgaaaatgtaaagattcctttagcatatatagactttgaaacttacattccacaattagaaaaagaacatgaacaatacaacaaagatattgcaaaagctgaaaaagatttagaaaagaaaccagataatcaaaaaacgattaataaaatagacaacttaaaacaacaaagagaagcaaatgaagctaaattagaagaagcacttcaactacaacaagaacatggtgatacattaccaatagcagctggtttctttattattaatccatttgaagttgtatattatgcaggtggttcatcgaatgaatatcgtcactttgcaggtagttatgcaattcagtgggaaatgattaaatacgcgttagatcacaacattgaccgttataacttctatggtatcagcggagacttctcagaagatgcacctgatgttggcgttattaaatttaaaaaaggttacaatgcagatgtttatgaatatattggtgatttcgttaaaccaattaataaaccagcgtacaaagcatatacaacactaaaaaaagtattaaaaaaataaatgattttcagtaagagaggaatttagataatatgaaatttacagagttaa

SEQ ID NO: 51 - protein, 410 aa, Staphylococcus sciuri femA
TLEFEAFTNKMPYAHFTQAVGNYELKTSEGTSTHLVGVKDNQGEVLAACLLTSVPVMKKFNYFYSNRGPVMDYDNKELVDFFFKEIVSYLKSYKGLFFRIDPYLPYQLRDHDGNIKKSFNRDGLIKQFESLGYEHQGFTTGFHPIHQIRWHSVLDLESMDEKTLIKNMDSLRKRNTKKVQKNGVKVRFLSKDEMPIFRQFMEDTTEKKDFNDRGDDFYYNRLKYFENVKIPLAYIDFETYIPQLEKEHEQYNKDIAKAEKDLEKKPDNQKTINKIDNLKQQREANEAKLEEALQLQQEHGDTLPIAAGFFIINPFEVVYYAGGSSNEYRHFAGSYAIQWEMIKYALDHNIDRYNFYGISGDFSEDAPDVGVIKFKKGYNADVYEYIGDFVKPINKPAYKAYTTLKKVLKK

SEQ ID NO: 52 - DNA, 1343 bp, Staphylococcus hominis femA
  CDS (64)...(1317)
taaaattttaaaattagtcaactcaaattaaataaagattctaaattaggagttatagagataatgaagtttacaaatttaacagctacagaatttggcgattttactgaaaaaatgccatatagccattttacacagatgactgaaaattatgagttaaaagttgctgagaaaactgaaactcatttagtaggaattaaaaataaagataatgaagtcattgctgcttgtatgctaactgctgtacccgttatgaaaatttttaaatatttttattcaaatcgtggtccagtcattgattatgaaaacaaagaactcgttcactttttctttaacgaattaagtaaatatttaaaacaacaacattgtttatatgtacgtatagacccttatttgccttatcaatatcgtaatcatgatggtgatattacaggaaatgctgggaatgattggttcttcgataaaatgaaacaattaggatatcaacacgaagggtttacaacaggatttgatccaatattacaaattcggttccattcagttttaaatttaaaggataaaactgctaaagatgtattaaatggaatggatagtttacgaaaaagaaatactaaaaaagtccaaaaaaatggtgttaaagtaagatttcttactaaagaagaattacctattttcagatcatttatggaagatacatcagagactaaagaattttctgatagagaggatagtttttactataatcgatttgatcattttaaagatagagtattagtacctctcgcatatataaaatttgatgaatatcttgaagaacttcatgcagaacgtcagacattaaataaagacttaaacaaagctctaaaagatattgaaaaacgaccagataacaaaaaagcacaaaataaaaaaataaatttagaacagcaattaaaagcaaatgagcaaaaaattgatgaagcaacacaacttcaattagaacatggtaacgaattaccaatatctgctggattcttctttattaatccatttgaagttgtatattatgcaggtggaacgtcaaataaatatagacacttcgctggaagttatgcagttcaatggactatgattaattatgcaattgatcatggcattgaccgttataatttttatgggattagtggtcattttacagatgatgctgaagatgcaggtgttgtaaaatttaaaaaaggatttaatgcagatgtaattgaatatgttggtgatttcgttaaacctataaataaaccaatgtattcactatatacaacacttaaaaaaattaaaaagagattgaattaagaggggaatagtgagaa

SEQ ID NO: 53 - protein, 418 aa, Staphylococcus hominis femA
MKFTNLTATEFGDFTEKMPYSHFTQMTENYELKVAEKTETHLVGIKNKDNEVIAACMLTAVPVMKIFKYFYSNRGPVIDYENKELVHFFFNELSKYLKQQHCLYVRIDPYLPYQYRNHDGDITGNAGNDWFFDKMKQLGYQHEGFTTGFDPILQIRFHSVLNLKDKTAKDVLNGMDSLRKRNTKKVQKNGVKVRFLTKEELPIFRSFMEDTSETKEFSDREDSFYYNRFDHFKDRVLVPLAYIKFDEYLEELHAERQTLNKDLNKALKDIEKRPDNKKAQNKKINLEQQLKANEQKIDEATQLQLEHGNELPISAGFFFINPFEVVYYAGGTSNKYRHFAGSYAVQWTMINYAIDHGIDRYNFYGISGHFTDDAEDAGVVKFKKGFNADVIEYVGDFVKPINKPMYSLYTTLKKIKKR

SEQ ID NO: 54 - DNA, 2742 bp, Staphylococcus saprophyticus femA
  CDS (64)...(1326)
acttgtttagattagaattaaactcgaaaatagaactatagataaataggagtatataaaaaaatgaaatttacgaatttaactgcaaaagagttcggtgcatttacggataaaatgccgaatagtcattttacgcaaatggttggaaattatgaattgaaaattgcagaaagtacagaaacacacctagtaggtattaagaataatgataatgaagtaattgcagcatgtttacttacagctgttcctgttatgaaattcttcaagtatttttattccaatagaggtccagtcatagattttgaaaataaagaactcgtacattacttctttaacgaattagcaaaatatgtaaaaaaacataatgccttatatttacgagtagatccttatcttgcttatcaatatcgtaatcatgatggtgaagtattagcaaatgcgggtcacgattggatttttgataaaatgaaacaactcggttataagcatgaaggttttttaactggctttgacccaatacttcaaataagattccattctgttttagatttagctggaaaaactgctaaagacgtacttaatggtatggatagtttacgtaaacgaaatactaaaaaagtacagaaaaatggtgtgaaagtaagatttttaggtgaagatgagttgccaatattccgctcattcatggaagatacttctgaaacaaaggattttgacgatagagatgacgatttttattataataggttaagatattataaagatcgtgtgcttgtcccattagcttatatggattttgatgaatatataacagaattaaaggctgaacgcgaagtattaagtaaagatataaataaagcagttaaggatatagaaaaaagaccagaaaataaaaaagcgtataataaaaaagaaaatttagaacaacaactgattgcaaaccaacaaaaaatagatgaagccactgcgttacaagagaagcatggtaacgaattaccgatttctgcagcttactttattattaatccttatgaagtcgtttactatgcaggtggtacatctaatgaatttagacattttgctggtagttatgcaatacaatggaagatgattaattatgctatagatcataatatagatagatataatttttatggtattagtggtcattttactgaagatgcagaagatgcaggtgttgttaaatttaaaaaaggttttaatgcagatgtagtagaatatgttggtgattttattaaaccgattaataagccaatgtacaaaatttatacgacattgaaaaaaattaaggataaaaagaaataaacataaatagaagggaactaagctagaatgaaatttacagagttaacttgtttagattagaattaaactcgaaaatagaactatagataaataggagtatataaaaaaatgaaatttacgaatttaactgcaaaagagttcggtgcatttacggataaaatgccgaatagtcattttacgcaaatggttggaaattatgaattgaaaattgcagaaagtacagaaacacacctagtaggtattaagaataatgataatgaagtaattgcagcatgtttacttacagctgttcctgttatgaaattcttcaagtatttttattccaatagaggtccagtcatagattttgaaaataaagaactcgtacattacttctttaacgaattagcaaaatatgtaaaaaaacataatgccttatatttacgagtagatccttatcttgcttatcaatatcgtaatcatgatggtgaagtattagcaaatgcgggtcacgattggatttttgataaaatgaaacaactcggttataagcatgaaggttttttaactggctttgacccaatacttcaaataagattccattctgttttagatttagctggaaaaactgctaaagacgtacttaatggtatggatagtttacgtaaacgaaatactaaaaaagtacagaaaaatggtgtgaaagtaagatttttaggtgaagatgagttgccaatattccgctcattcatggaagatacttctgaaacaaaggattttgacgatagagatgacgatttttattataataggttaagatattataaagatcgtgtgcttgtcccattagcttatatggattttgatgaatatataacagaattaaaggctgaacgcgaagtattaagtaaagatataaataaagcagttaaggatatagaaaaaagaccagaaaataaaaaagcgtataataaaaaagaaaatttagaacaacaactgattgcaaaccaacaaaaaatagatgaagccactgcgttacaagagaagcatggtaacgaattaccgatttctgcagcttactttattattaatccttatgaagtcgtttactatgcaggtggtacatctaatgaatttagacattttgctggtagttatgcaatacaatggaagatgattaattatgctatagatcataatatagatagatataatttttatggtattagtggtcattttactgaagatgcagaagatgcaggtgttgttaaatttaaaaaaggttttaatgcagatgtagtagaatatgttggtgattttattaaaccgattaataagccaatgtacaaaatttatacgacattgaaaaaaattaaggataaaaagaaataaacataaatagaagggaactaagctagaatgaaatttacagagtta

SEQ ID NO: 55 - protein, 420 aa, Staphylococcus saprophyticus femA
MKFTNLTAKEFGAFTDKMPNSHFTQMVGNYELKIAESTETHLVGIKNNDNEVIAACLLTAVPVMKFFKYFYSNRGPVIDFENKELVHYFFNELAKYVKKHNALYLRVDPYLAYQYRNHDGEVLANAGHDWIFDKMKQLGYKHEGFLTGFDPILQIRFHSVLDLAGKTAKDVLNGMDSLRKRNTKKVQKNGVKVRFLGEDELPIFRSFMEDTSETKDFDDRDDDFYYNRLRYYKDRVLVPLAYMDFDEYITELKAEREVLSKDINKAVKDIEKRPENKKAYNKKENLEQQLIANQQKIDEATALQEKHGNELPISAAYFIINPYEVVYYAGGTSNEFRHFAGSYAIQWKMINYAIDHNIDRYNFYGISGHFTEDAEDAGVVKFKKGFNADVVEYVGDFIKPINKPMYKIYTTLKKIKDKKK

SEQ ID NO: 56 - DNA, 18 bp, Artificial Sequence
  Primer
ttcmaatcgcggtccagt

SEQ ID NO: 57 - DNA, 23 bp, Artificial Sequence
  Primer
caagaacatggcaacgaattacc

SEQ ID NO: 58 - DNA, 23 bp, Artificial Sequence
  Primer
tgggtaattcgttgccatgttct

SEQ ID NO: 59 - DNA, 21 bp, Artificial Sequence
  Primer
ccaagcatcttcagcatcttc

SEQ ID NO: 60 - DNA, 29 bp, Artificial Sequence
  Primer
ttctttaactgttaactctgtaaatttca

SEQ ID NO: 61 - DNA, 26 bp, Artificial Sequence
  Primer
acatatttacttaattcgttaaagaa

SEQ ID NO: 62 - DNA, 27 bp, Artificial Sequence
  Primer
cagaaaaatggtgttaaagtaagattt

SEQ ID NO: 63 - DNA, 27 bp, Artificial Sequence
  Primer
aagaaatcttactttcacaccattttt

SEQ ID NO: 64 - DNA, 18 bp, Artificial Sequence
  Primer
aactcgaaaatagaacta

SEQ ID NO: 65 - DNA, 20 bp, Artificial Sequence
  Universal primer
gccatacagtcatttcacgc

SEQ ID NO: 66 - DNA, 18 bp, Artificial Sequence
  femA primer for identification of S. aureus
cagcagatgacatcatta

SEQ ID NO: 67 - DNA, 18 bp, Artificial Sequence
  femA primer for identification of S. haemolyticus
aaccgaaacgaaagaatt

SEQ ID NO: 68 - DNA, 21 bp, Artificial Sequence
  femA primer for identification of S. hominis
caacacaacttcaattagaac

SEQ ID NO: 69 - DNA, 27 bp, Artificial Sequence
  primer/probe for identification of S. aureus sbs. anaerobius
atttaaaatatcacgctcttcgtttag

SEQ ID NO: 70 - DNA, 27 bp, Artificial Sequence
  primer/probe for identification of S. epidermidis
attaagcacatttctttcattatttag

SEQ ID NO: 71 - DNA, 27 bp, Artificial Sequence
  primer/probe for identification of S. haemolyticus
atttaaagtttcacgttcattttgtaa

SEQ ID NO: 72 - DNA, 27 bp, Artificial Sequence
  primer/probe for identification of S. hominis
atttaatgtctgacgttctgcatgaag

SEQ ID NO: 73 - DNA, 27 bp, Artificial Sequence
  primer/probe for identification of S. saprophyticus
acttaatacttcgcgttcagcctttaa

SEQ ID NO: 74 - DNA, 27 bp, Artificial Sequence
  primer/probe for identification of S. capitis
attaagaacatctctttcattattaag

SEQ ID NO: 75 - DNA, 27 bp, Artificial Sequence
  primer/probe for identification of S. cohniisbs. urealyticum
acttaacacttcacgctctgacttgag

SEQ ID NO: 76 - DNA, 27 bp, Artificial Sequence
  primer/probe for identification of S. gallinarum
acttaaaacttcacgttcagcagtaag

SEQ ID NO: 77 - DNA, 27 bp, Artificial Sequence
  primer/probe for identification of S. intermedius
gtggaaatcttgctcttcagatttcag

SEQ ID NO: 78 - DNA, 27 bp, Artificial Sequence
  primer/probe for identification of S. lugdunensis
ttctaaagtttgtcgttcattcgttag

SEQ ID NO: 79 - DNA, 27 bp, Artificial Sequence
  primer/probe for identification of S. schleiferi
tttaaagtcttgcgcttcagtgttgag

SEQ ID NO: 80 - DNA, 27 bp, Artificial Sequence
  primer/probe for identification of S. sciuri
gttgtattgttcatgttctttttctaa

SEQ ID NO: 81 - DNA, 27 bp, Artificial Sequence
  primer/probe for identification of S. simulans
ttctaaattcttttgttcagcgttcaa

SEQ ID NO: 82 - DNA, 27 bp, Artificial Sequence
  primer/probe for identification of S. warneri
agttaaggtttctttttcattattgag

SEQ ID NO: 83 - DNA, 27 bp, Artificial Sequence
  primer/probe for identification of S. xylosus
gcttaacacctcacgttgagcttgcaa

(19) United States
(12) Patent Application Publication
(10) Pub. No.: US 2004/0146905 A1
(43) Pub. Date: Jul. 29, 2004

(54) Genetic sequences, diagnostic and/or quantification methods and devices for the identification of staphylococci strains

(76) Inventors: Pascal Vannuffel, Buvrinnes (BE); Jean-Luc Gala, St. Stevens-Woluwe (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/703,649

(22) Filed: Nov. 7, 2003

Related U.S. Application Data
(63) Continuation-in-part of application No. 09/509,234, filed on Sep. 25, 2000, now abandoned.

(30) Foreign Application Priority Data
Sep. 28, 1998 (WO) PCT/BE98/00141
Sep. 26, 1997 (BE) EP97870146.4

(51) Int. Cl. C12Q 1/68; C07H 21/04

(52) U.S. Cl. 435/6; 536/23.7

(57) ABSTRACT

The present invention is related to oligonucleotides for the specific identification of Staphylococci species which nucleotide sequence has between 15 and 350 base pairs, preferably between 15 and 45 base pairs, obtained from the “consensus” femA nucleotide sequence (CNS) of FIG. 3 or its complementary strand. The present invention is also related to a method and a diagnostic device using said oligonucleotide for the identification of various types of Staphylococci species strains.

This patent application is a continuation-in-part (CIP) of U.S. Ser. No. 09/509,234, filed Sep. 25, 2000, the contents of all of which are incorporated by reference into the present application.

### FIELD OF THE INVENTION

The present invention refers to new genetic sequences, diagnostic and/or quantification methods and devices using said sequences for the identification of various types of Staphylococci strains as well as the therapeutical aspects of said genetic sequences.

### BACKGROUND OF THE INVENTION

Increasing incidence of nosocomial infections by multiresistant bacteria (even to antibiotics like vancomycin) is a world-wide concern. Methicillin-resistant coagulase-negative Staphylococci (MR-CNS) and S. aureus (MRSA) express a high level cross-resistance to all β-lactam antibiotics (Ryffel et al. (1990), Refsahl et al. (1992)). They have an additional low-affinity penicillin-building protein, PBP2a (PBP2′), encoded by the mecA gene. The mecA determinant is found in all multiresistant staphylococcal species (Chackbart et al. (1989), Suzuki et al. (1992), Vannuffel et al. (1995)) and is highly conserved among the different species (Ryffel et al. (1990)).

Several other chromosomal sites, in which transposon inactivation reduces the level of β-lactam resistance, have been identified in S. aureus (SA) (Hiramatsu (1992), Berger-Bachi et al. (1992), de Lancastre et al. (1994)). The appropriate functioning of these regulator genes rather than the quantity of PBP2a determines the minimal inhibitory concentration value and homogeneous expression of resistance of staphylococcal isolates (Ryffel et al. (1994), de Lancastre et al. (1994)).

The femA-femB operon, initially identified in S. aureus, is one of those genetic factors essential for methicillin resistance (Berger-Bächi et al. (1989)). It is involved in the formation of the characteristic pentaglycine side chain of the SA peptidoglycan (Stranden et al. (1997)). Unlike other regulatory genes, femA was shown to retain a strong conservation over time in clinical isolates of MRSA, hence confirming its key role in cell wall metabolism and methicillin resistance (Hurlimann-Dalel et al. (1992)). In contrast to mecA, femA-femB is present both in the genome of resistant and susceptible SA strains (Unal et al. (1992), Vannuffel et al. (1995)).

Often, identification of the Staphylococci is limited to a rapid screening test for S. aureus, and non-S. aureus isolates are simply reported as coagulase-negative Staphylococci. In fact, these bacteria isolates include a variety of species and many different strains (Kleeman et al. (1993)). There is little epidemiological information related to the acquisition and spread of these organisms. This is potentially due to the lack of an easy and accurate way to identify species and to provide clinically timely informations.

Several molecular assays designed for detecting femA in SA failed to amplify an homologous sequence in coagulase-negative Staphylococci (Kizaki et al. (1994), Vannuffel et al. (1995)). Nevertheless, low-stringency heterologous hybridisation analysis suggested the presence of such a structurally related gene in S. epidermidis (SE) (Unal et al. (1992)).

These data were followed by complete identification and sequence analysis of the femA and femB open reading frames in S. epidermidis (Albom et al. (1996)). Intra- and interspecies relatedness of these genes and conservation of genomic organisation are therefore consistent with gene duplication of one of these genes in an ancestral organism and the possibility of femA phylogenetic conservation in all staphylococcal species (Alborn et al. (1996)).

The complete genetic sequence of the femA gene de S. epidermidis, the protein encoded by the femA gene (FemA) and vectors and micro-organisms comprising genes encoding the FemA protein are described in the U.S. Pat. No. 5,587,307.

Aims of the Invention

The present invention aims to provide new genetic sequences, methods and devices for the improvement of the identification and/or the quantification of various types of Staphylococci strains through their femA-like determinants, which allow by a rapid screening their epidemiological study.

Another aim of the invention is to identify similar genetic sequences which may exist in known or not known Staphylococci species or other gram-positive bacterial strains.

A last aim of the present invention is to provide new sequences encoding femA proteins of Staphylococci species, their femA proteins, vector(s) comprising said nucleotide sequences and cell (s) transformed by said vector(s) for possible therapeutical applications.

### SUMMARY OF THE INVENTION

The Inventors have identified new DNA and amino acid sequences from new strains of Staphylococcus hominis, Staphylococcus saprophyticus and Staphylococcus haemolyticus. Said new nucleotide sequences allow an alignment of these new sequences with the femA gene from Staphylococci previously described (S. aureus, S. epidermidis and S. saprophyticus). By the alignment of more than 2 sequences, preferably more than 4 sequences, the Inventors have identified for the first time a consensus femA sequence useful for molecular genotyping of different Staphylococci species which was not possible previously, when only few femA sequences of Staphylococci strains were known.

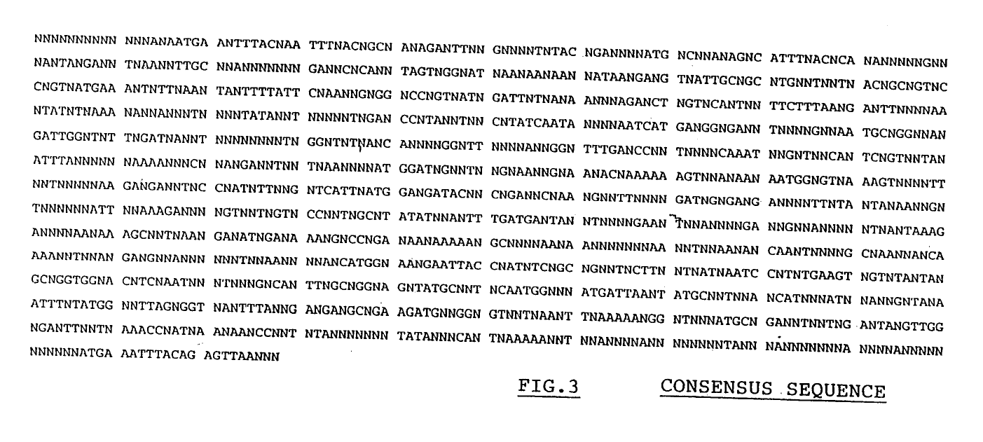

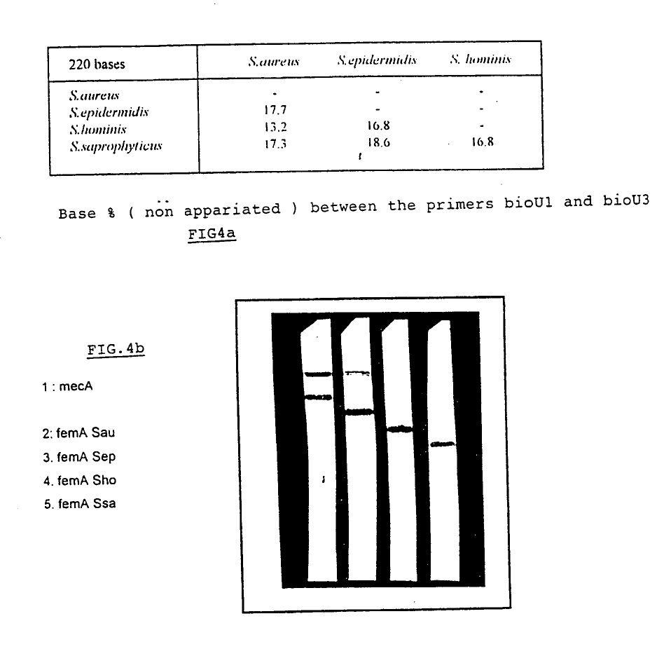

Therefore, a first aspect of the present invention is related to the “consensus” nucleotide sequence as represented in the enclosed FIG. 3. With said “consensus” nucleotide sequence, the Inventors were able to provide oligonucleotides (such as primers or probes) which can be used for the genetic amplification, the identification and/or quantification of various femA sequences which are specific of known or unknown Staphylococci species.

The femA sequence is known to be involved with the biosynthesis of glycin-containing cross-bridges of the peptidoglycan and the peptidoglycan organisation is also known to be well conserved among various Staphylococci species and possibly among other gram-positive bacteria.

Therefore, it is also possible to use the new “consensus” femA sequence and said new oligonucleotides extrapolated from the alignment of the sequences presented in FIG. 3, for the molecular genotyping of other Staphylococci species and possibly other gram-positive bacteria. It is also known that the femA sequence is similar to the femB sequence. Therefore, these oligonucleotides could also be used for the molecular genotyping of femB genes of different Staphylococci species or other gram-positive bacteria.

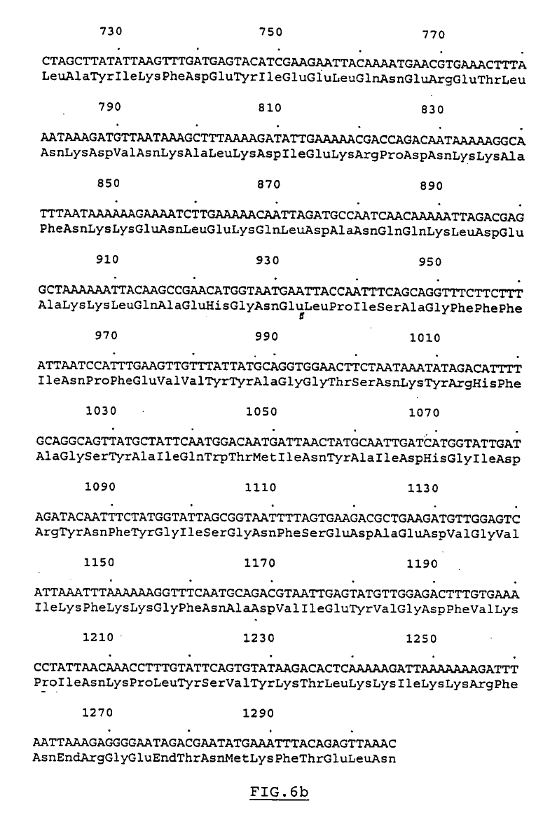

Another aspect of the present invention concerns the possible therapeutical uses of new femA nucleotide sequences isolated from the strains S. hominis, S. saprophyticus, S. haemolyticus, S. lugdunensis, S. xylosus, S. capitis, S. schleiferi and S. sciuri having a nucleotide or amino acid sequence which presents more than 85%, preferably more than 90% homology or 100% homology with the genetic sequences presented in the FIGS. 6 to 13, their complementary strand and functional variants thereof. Functional variants of said amino acid sequences are peptides which contain one or more modifications to the primary amino acids sequence and retain the activity of the complete and wild type femA molecule. Variants of the peptide are obtained by nucleotidic sequences which differ from the above-identified described sequences by a degeneration of their genetic code or are sequences which hybridise with said sequences or their complementary strand, preferably under stringent conditions such as the ones described in the document Sambrook et al., §§ 9.47-9.51 in Molecular Cloning: A Laboratory Manual, Cold Spring Harbor, Laboratory Press, Cold Spring Harbor, N.Y. (1989).

A further aspect of the present invention concerns the recombinant vector (i.e. constructions into which the sequence of the invention may be inserted for transport in different genetic environments and for expression in a host cell, such as a phagemide, a virus, a plasmid, a cationic vesicle, a liposome, etc.) comprising said nucleotide sequences and their complementary strands, or the corresponding RNA sequences, possibly linked to one or more regulatory sequences or markers (resistance to antibiotics, enzyme coding sequences, . . . ) active into a cell.

Similarly, the nucleic acid sequence according to the invention may be obtained by synthetic methodology well known by the person skilled in the art, such as the one described by Brown et al. (“Method of Enzymology”, Acad. Press, New-York, No. 68 pp. 109-151 (1979)) or by conventional DNA synthesising apparatus such as the applied biosystem model 380A or 380B DNA synthesiser.

Other aspects of the present invention concern the recombinant host (prokaryotic) cell transformed by said vector and the purified (possibly recombinant) proteins or peptides encoded by said nucleic acid sequences, possibly linked to a carrier molecule such as BSA and obtained by said cells. Said recombinant proteins or peptides could be obtained by genetic engineering or could be obtained by synthesis (see U.S. Pat. No. 5,587,307 incorporated herein by reference) and may comprise residues enhancing their stability (resistance to hydrolysis by proteases, etc.) such as the one described by Nachman et al. (Regul. Pept. Vol. 57, pp. 359-370 (1995)).

A preferred vector for expression in a E. coli host cell is derived from the E. coli plasmid pET-11A available from Novagen Inc. (Catalogue No. 69436-A). The transformation technique used with the above-identified vector has been described in the U.S. Pat. No. 5,587,307.

A further aspect of the present invention concerns the inhibitor (used to possibly treat (with addition of antibiotics) antibiotics resistance bacteria) directed against said proteins, peptides or nucleic acid molecules. Advantageously, said inhibitor is a antibody, preferably a monoclonal antibody, or an antisense nucleotide molecule, such as a ribozyme, which could be present in a vector in order to block the expression of said femA nucleotide sequences.

A last aspect of the present invention concerns the pharmaceutical composition, preferably a vaccine, against Staphylococci infections in an animal, including a human, comprising a pharmaceutically acceptable carrier and a sufficient amount of an active compound selected from the group consisting of said nucleic acid molecules, vectors, recombinant host cells transformed by said vector(s), inhibitors (directed against said proteins, peptides or nucleic acid molecules) and a mixture thereof.

Another aspect of the present invention concerns oligonucleotides which are (DNA) sequences having between 15 and 350 base pairs, preferably between 17 and 250 base pairs (such as primers or probes) obtained from the consensus sequence of FIG. 3 or its complementary strand. Preferably, said oligonucleotides are primers having between 15 and 45 base pairs, more preferably between 17 and 25 base pairs.

According to a first embodiment of the present invention, said oligonucleotide is a primer having between 15 and 45 base pairs, which presents more than 60%, advantageously more than 70%, preferably more than 80%, more specifically more than 90% homology with (fragments of) the “consensus” femA nucleotide sequence (CNS) identified in the FIG. 3.

Therefore, the oligonucleotides according to the invention are new sequences or preferred fragments of known sequences of S. aureus, S. epidermidis or S. simulans but not the complete wild type known femA nucleotide sequence.

Preferably, the oligonucleotide according to the invention is selected from the group consisting of the following nucleotide sequences:

ANAATGAANTTTACNAATTTNACNGCNANAGANTT (SEQ ID NO: 2) and more particularly femS1 TAATGAAGTTTACAAAATTT (SEQ ID NO: 3) or femS2 TAATGAAGTTTACNAAATTT (SEQ ID NO: 4)

ATGNCNNANAGNCATTTNACNCANA (SEQ ID NO: 5) and more particularly femU1 (“universal” sequence sense of the multiplex PCR): TGCCATATAGTCATTTACGC (SEQ ID NO: 6)

TAGTNGGNATNAANAANAANNATAANGANGTNATTGC (SEQ ID NO: 7)

GTNCCNGTNATGAAANTNTTNAANTANTTTTATTC (SEQ ID NO: 8)

AATGCNGGNNANGATTGG (SEQ ID NO: 9)

GNAANNGNAANACNAAAAAAGTNNANAANAATGGNGTNAAAGT (SEQ ID NO: 10) and more particularly fsq1S (et 1AS): AAAAAGTTCAAAAAATGG (SEQ ID NO: 11) and fsq2S (and 2AS): AAAAAGTACAAAAAATGG (SEQ ID NO: 12)

AAGANGANNTNCCNATNTTNNGNTCATTNATGGANGATAC (SEQ ID NO: 13)

TATATNNANTTTGATGANTA (SEQ ID NO: 14)

AANGANATNGANAAANGNCCNGANAANAAAAA (SEQ ID NO: 15) and more particularly fsq3S (and 3AS): AAAGATATTGAAAAACGA (SEQ ID NO: 16), fsq4S (and 4AS): AAAGATATTGAAAAGAGACC (SEQ ID NO: 17), fsq5S (and 5AS): AAAGATATCGAGAAAGAC (SEQ ID NO: 18) and fsq6S (and 6AS) AAAGACATCGACAAGCGT (SEQ ID NO: 19).

ANCATGGNAANGAATTACCNAT (SEQ ID NO: 20) and more particularly fem1 (primer for the production of a probe and of marked amplicons for reverse hybridisation experiment): GAACATGGTAATGAATTAC (SEQ ID NO: 21)

AATCCNTNTGAAGTNGTNTANTANGCNGGTGG (SEQ ID NO: 22)

AGNTATGCNNTNCAATGGNNNATGATTAANTATGC (SEQ ID NO: 23)

TTTANNGANGANGCNGAAGATGNNGGNGTNNTNAANTTNAAAAA (SEQ ID NO: 24) and more particularly fem3bio (primer for the production of a probe and of marked amplicons for reverse hybridization experiment): TTTACTGAAGATGCTGAAGA (SEQ ID NO: 25)

GTTGGNGANTTNNTNAAACC (SEQ ID NO: 26) and more particularly fem2 (primer for the production of a probe and of marked amplicons for reverse hybridisation experiment): GTTGGTGACTTTATTAAACC (SEQ ID NO: 27)

ATGAAATTTACAGAGTTAA (=femAS1) (SEQ ID NO: 28).

In addition, the following universal primer may also be used in the present invention: GCCATACAGTCATTTCACGC (=uni) (SEQ ID NO: 65).

Said primer(s) will be designated hereafter as “universal primer(s)”.

A further aspect of the present invention concerns the oligonucleotide being either a primer or a probe as above-described, having between 15 and 350 base pairs, preferably between 17 and 250 base pairs, or a primer having between 15 and 45 base pairs, more preferably between 17 and 25 base pairs, which will be designated hereafter as “specific primer(s)”, having a nucleotide sequence which presents less than 50%, advantageously less than 40%, preferably less than 30%, more specifically less than 20% homology with (fragments of) the “consensus” femA nucleotide sequence (CNS) identified in the FIG. 3 and with another femA nucleotide sequence specific for other Staphylococci strains.

Advantageously, said “specific primer” is selected from the group consisting of the following nucleotide sequences:

| | |
|---|---|
| ACAGCAGATGACATCATT | (SEQ ID NO: 29) |
| TAATGAAAGAAATGTGCTTA | (SEQ ID NO: 30) |
| ACACAACTTCAATTAGAAC | (SEQ ID NO: 31) |
| AGTATTAGCAAATGCGG | (SEQ ID NO: 32) |
| ATGCATATTTTCCGTAA | (SEQ ID NO: 33) |
| CAGCAGATGACATCATT | (SEQ ID NO: 34) |
| CATCTAAAGATATATTAAATGGA | (SEQ ID NO: 35) |
| AGTATTAGCAAATGCGGGTCAC | (SEQ ID NO: 36) |
| CAACACAACTTCAATTAGAA | (SEQ ID NO: 37) |

In addition, the following specific femA primers can be used in a multiplex protocol for staphylococcal differential diagnosis:

| | |
|---|---|
| CAGCAGATGACATCATTA | (SEQ ID NO: 66) |
| for identification of S. aureus, | |
| AACCGAAACGAAAGAATT | (SEQ ID NO: 67) |
| for identification of S. haemolyticus, | |
| and | |
| CAACACAACTTCAATTAGAAC | (SEQ ID NO: 68) |
| for identification of S. hominis. | |

Further, the following primers or probes can be used for each of the following staphylococcal species, in the method or kit according to the present invention for the identification and/or quantification of a Staphylococci species, which may present resistance to antibiotics and which is present in a sample:

| | | |
|---|---|---|
| S. aureus sbs. anaerobius: | ATTTAAAATATCACGCTCTTCGTTTAG, | (SEQ ID NO:69) |
| S. epidermidis: | ATTAAGCACATTTCTTTCATTATTTAG, | (SEQ ID NO:70) |
| S. haemolyticus: | ATTTAAAGTTTCACGTTCATTTTGTAA, | (SEQ ID NO:71) |
| S. hominis: | ATTTAATGTCTGACGTTCTGCATGAAG, | (SEQ ID NO:72) |
| S. saprophyticus: | ACTTAATACTTCGCGTTCAGCCTTTAA, | (SEQ ID NO:73) |
| S. capitis: | ATTAAGAACATCTCTTTCATTATTAAG, | (SEQ ID NO:74) |
| S. cohnii sbs. urealyticum: | ACTTAACACTTCACGCTCTGACTTGAG, | (SEQ ID NO:75) |
| S. gallinarum: | ACTTAAAACTTCACGTTCAGCAGTAAG, | (SEQ ID NO:76) |
| S. intermedius: | GTGGAAATCTTGCTCTTCAGATTTCAG, | (SEQ ID NO:77) |
| S. lugdunensis: | TTCTAAAGTTTGTCGTTCATTCGTTAG, | (SEQ ID NO:78) |
| S. schielferi: | TTTAAAGTCTTGCGCTTCAGTGTTGAG, | (SEQ ID NO:79) |
| S. sciuri: | GTTGTATTGTTCATGTTCTTTTTCTAA, | (SEQ ID NO:80) |
| S. simulans: | TTCTAAATTCTTTTGTTCAGCGTTCAA, | (SEQ ID NO:81) |
| S. warneri: | AGTTAAGGTTTCTTTTTCATTATTGAG, | (SEQ ID NO:82) |
| S. xylosus: | GCTTAACACCTCACGTTGAGCTTGCAA. | (SEQ ID NO:83) |

The oligonucleotides according to the invention are selected according to their physiochemical properties in order to avoid cross-hybridisation between themselves. Said primers are not complementary to each other and they contain a similar percentage of bases GC.

Said oligonucleotides are used in an identification and/or quantification method of one or more Staphylococcus species and possibly other gram-positive bacteria. In a preferred embodiment, said oligonucleotides are used in an identification and/or quantification method of at least two different Staphylococcus species and possibly other gram-positive bacteria.

Therefore, another aspect of the present invention is related to an identification and/or quantification method of a Staphylococci species which may present resistance to one or more antibiotic(s), and is possibly combined with a method for the identification of a resistance to antibiotics, especially β-lactam antibiotics, (for instance through the identification of a variant of the mecA gene as described by Vannuffel et al. (1998)).

In another embodiment, the present invention is related to an identification and/or quantification method of at least two different Staphylococci species. The diagnostic device for the identification of at least two Staphylococci species comprises preferably at least one oligonucleotide which has more than 60% homology to SEQ ID NO:1 and at least one isolated or purified oligonucleotides which has less than 50% homology to SEQ ID NO:1.

The method for the detection, the identification and/or the quantification of a bacteria, preferably a staphylococcal species, comprises the steps of:

obtaining a nucleotide sequence from said bacteria present in a sample, preferably a biological body sample obtained from a patient such as blood, serum, dialyse liquid or cerebrospinal liquid, or from any other bacteriological growth medium,

possibly purifying said nucleotide sequence from possible contaminants,

possibly amplifying by known genetic amplification techniques said nucleotide sequence with one or more universal oligonucleotide(s) (universal primer(s)) according to the invention, and

identifying the specific gram-positive bacteria species, preferably the specific Staphylocossi species:

by a comparative measure of the length of the (possibly amplified) nucleotide sequence or

by reverse hybridisation of the (possibly amplified) nucleotide sequence with one or more specific oligonucleotide(s) (specific probe(s) or primer(s)) according to the invention which are specific of said bacteria, said oligonucleotide(s) being preferably immobilised on a solid support.

The comparative measure of the length of a possibly amplified nucleotide sequences can be performed by the analysis of their migration (compared with a known ladder) upon an electrophoresis gel.

Preferably, the genetic amplification technique is selected from the group consisting of PCR (U.S. Pat. No. 4,965,188), LCR (Landgren et al., Sciences, 241, pp. 1077-1080 (1988)), NASBA (Kievits et al., J. Virol. Methods, 35, pp. 273-286 (1991)), CPR (patent WO95/14106) or ICR.

The specific detection of the possibly amplified nucleotide sequences can be obtained by the person skilled in the art by using known specific gel electrophoresis techniques, in situ hybridisation, hybridisation on solid support, in solution, on dot blot, by Northern blot or Southern blot hybridisation, etc.

Advantageously, the probes which are specific of the bacteria are immobilised on a solid support according to the method described in the international patent application WO98/11253 incorporated herein by reference.

Said specific oligonucleotides (probes or “elongated” primers) have a length comprised between 50 and 350 base pairs, preferably between 120 and 250 base pairs, and are fixed to the solid support by a terminal 5′ phosphate upon an amine function of the solid support by carbodiimide reaction (as described in the document WO98/11253 incorporated herein by reference).

The solid support can be selected from the group consisting of cellulose or nylon filters, plastic supports such as 96-well microtiter plates, microbeads, preferably magnetic microbeads, or any other support suitable for the fixation of a nucleotide sequence.

The method according to the invention can be advantageously combined with another specific detection step of a possible resistance to antibiotics, especially β-lactam antibiotics (for instance through the identification by the above-described technique of variants of the mecA gene as described by Vannuffel et al. (1998)).

The present invention concerns also a diagnostic and/or quantification device or kit for the identification and/or the quantification of a Staphylococcus species or other gram-positive bacteria, comprising the oligonucleotides according to the invention and possibly all the media necessary for the identification of a (possibly amplified) nucleotide sequence of said bacteria through any one of the above-described methods. In another embodiment, the present invention concerns also a diagnostic and/or quantification device or kit for the identification and/or the quantification of at least two different Staphylococcus species or other gram-positive bacteria, comprising the oligonucleotides according to the invention and possibly all the media necessary for the identification of a (possibly amplified) nucleotide sequence of said bacteria through any one of the above-described methods.

Advantageously, the method and device according to the invention are adapted for the quantification of said Staphylococci strains by the use of a “internal or external standard sequence”, preferably the one described in the patent application WO98/11253 incorporated herein by reference.

Therefore, according to a first embodiment of the present invention, the nucleic acid sequence from a Staphylococcus species, for instance Staphylococcus aureus, is amplified by a “universal primer” and by a “specific primer” which is specific for S. aureus. The identification of S. aureus will be obtained upon an agarose electrophoresis gel wherein the amplified nucleotide sequence (shorter than the amplified nucleotide sequence of another Staphylococci species such as S. epidermidis) and identified by the use of a comparative ladder.

According to another embodiment of the present invention, a Staphylococcus species (such as S. aureus) is identified by reverse hybridisation of the amplified nucleotide sequence with a probe which is specific of said bacteria and which is immobilised on a solid support such as filter.

The present invention will be described in details in the following non-limiting examples, in reference to the Figures described hereafter.

### SHORT DESCRIPTION OF THE DRAWINGS

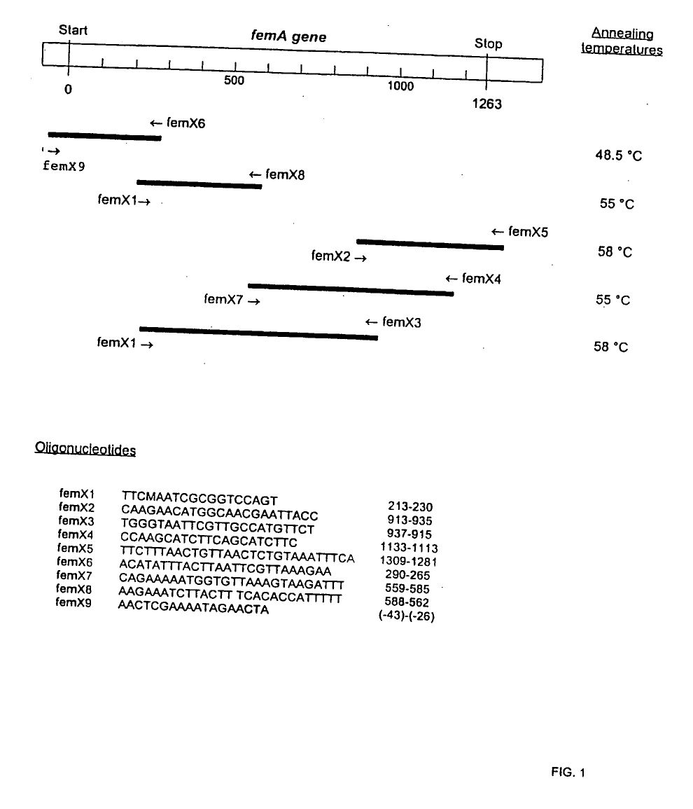

The FIG. 1 represents 5 partially overlapping fragments of the femA genes from S. hominis, S. saprophyticus and S. haemolyticus obtained by PCR amplification.

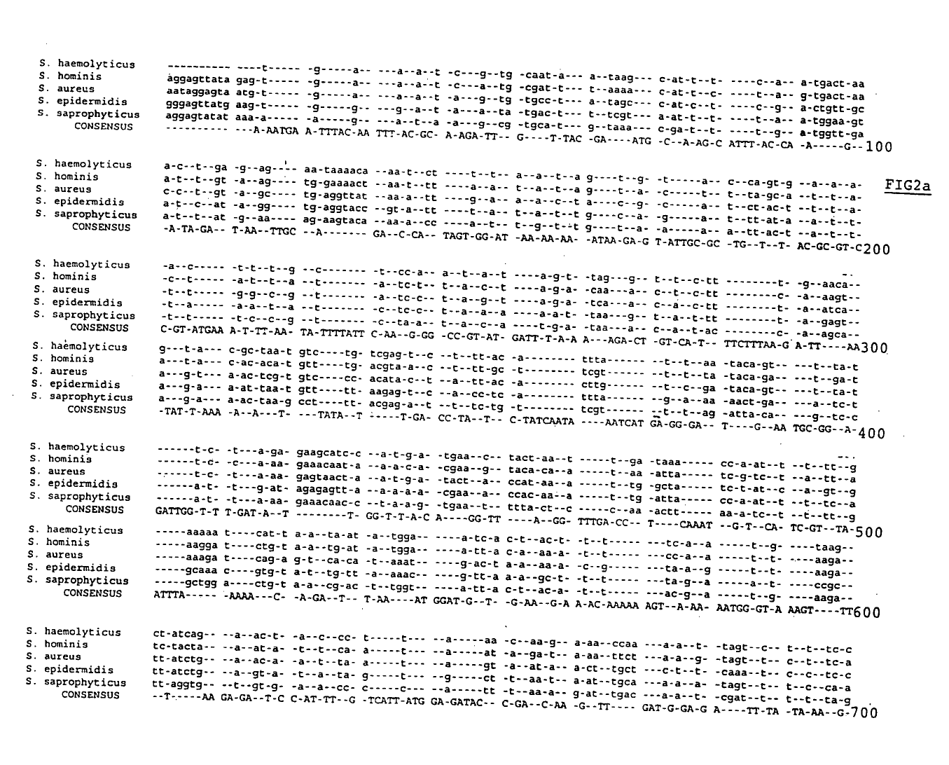

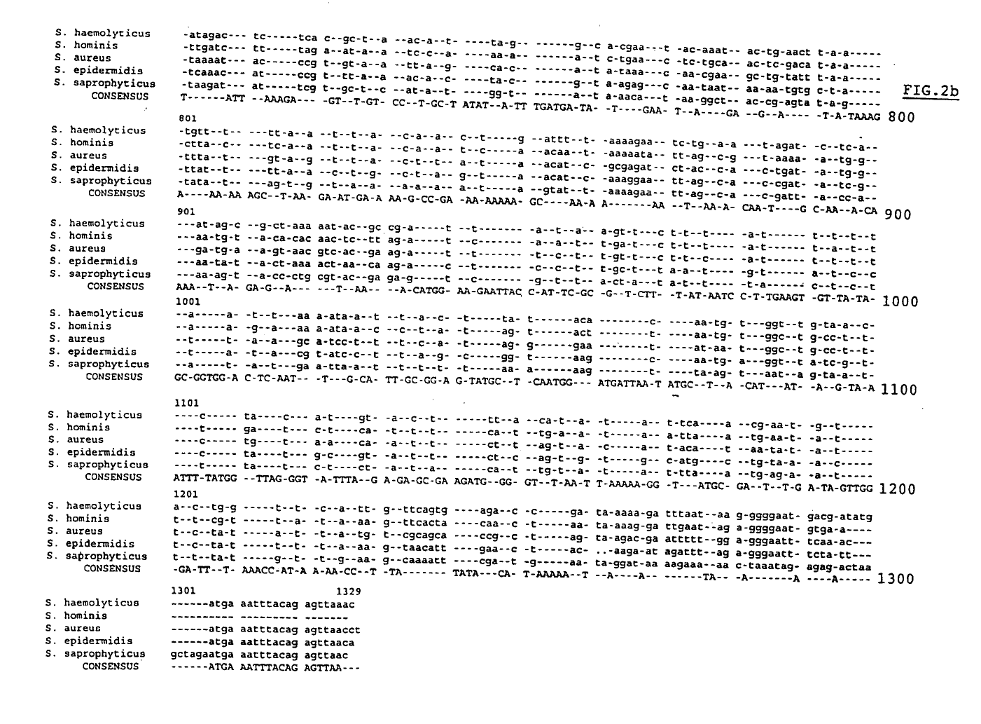

The FIGS. 2a & b represent the alignment of the nucleotide sequences of femA genes from S. hominis, S. saprophyticus, S. aureus, S. epidermidis and S. haemolyticus.

The FIG. 3 represents the consensus sequence according to the invention.

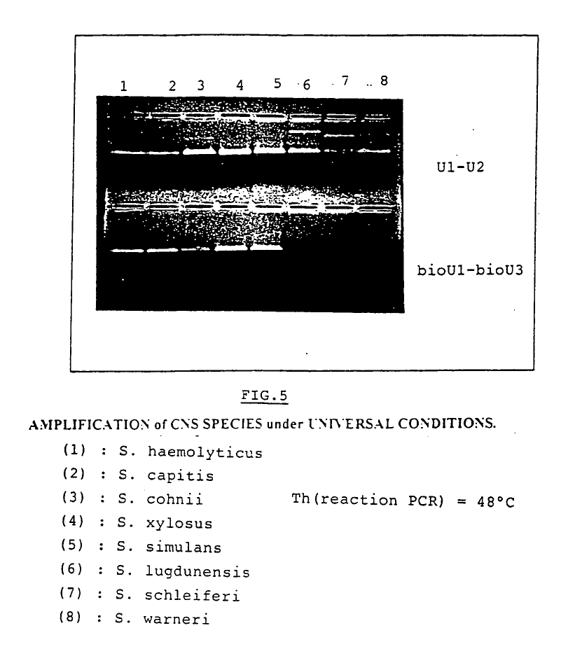

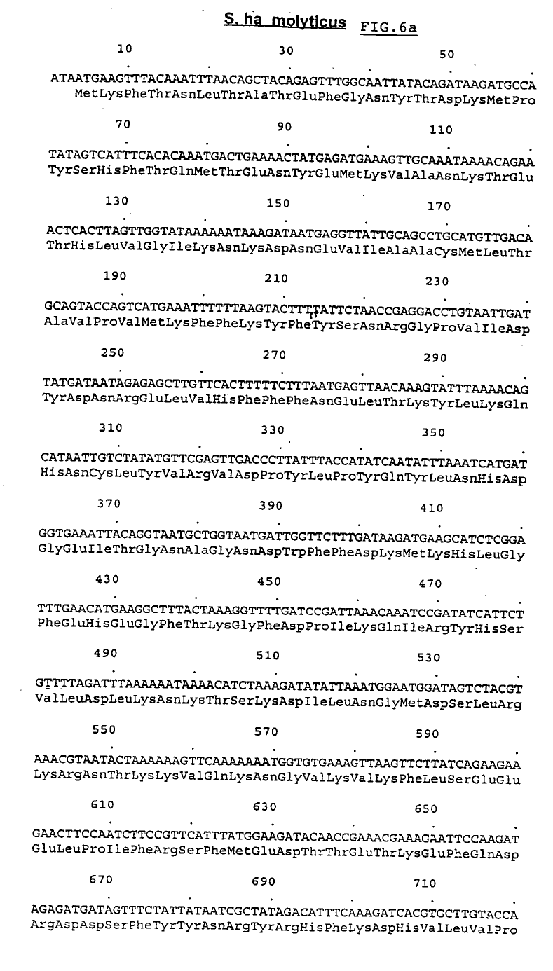

The FIG. 4 represents the result of differential diagnosis between different strains of Staphylococci by reverse hybridisation.

The FIG. 5 represents amplification of CNS species under universal conditions.

The FIGS. 6a & b represent the complete femA wild type genetic sequence of the strain S. haemolyticus.

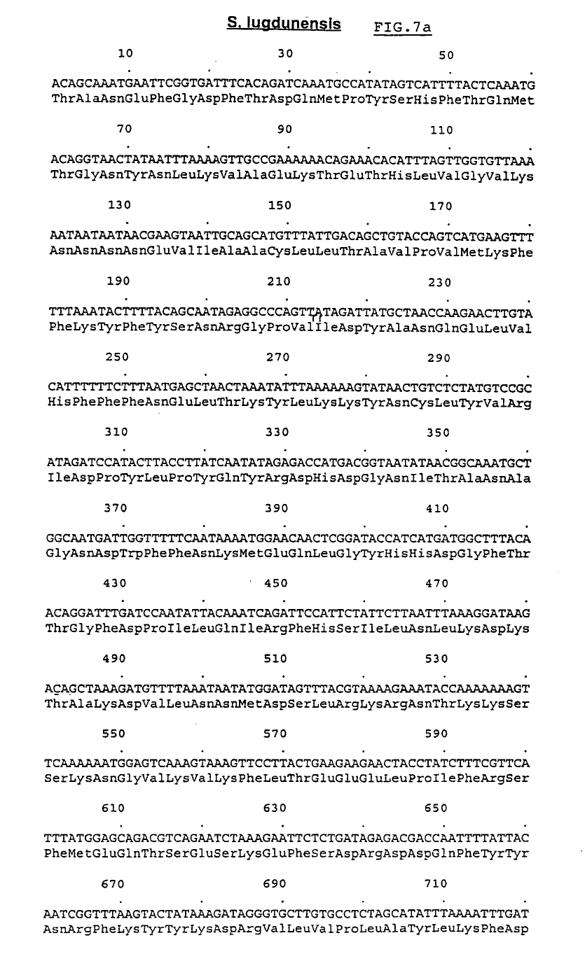

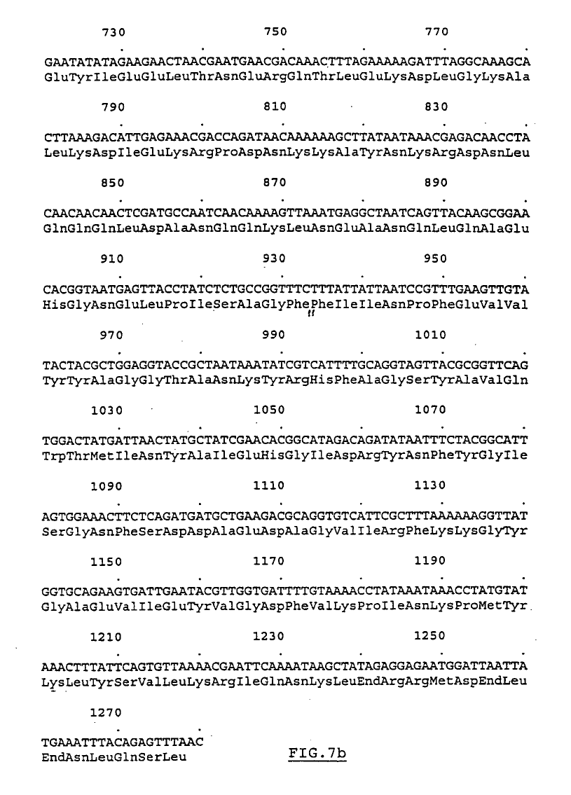

The FIGS. 7a & b represent the complete femA wild type genetic sequence of the strain S. lugdunensis.

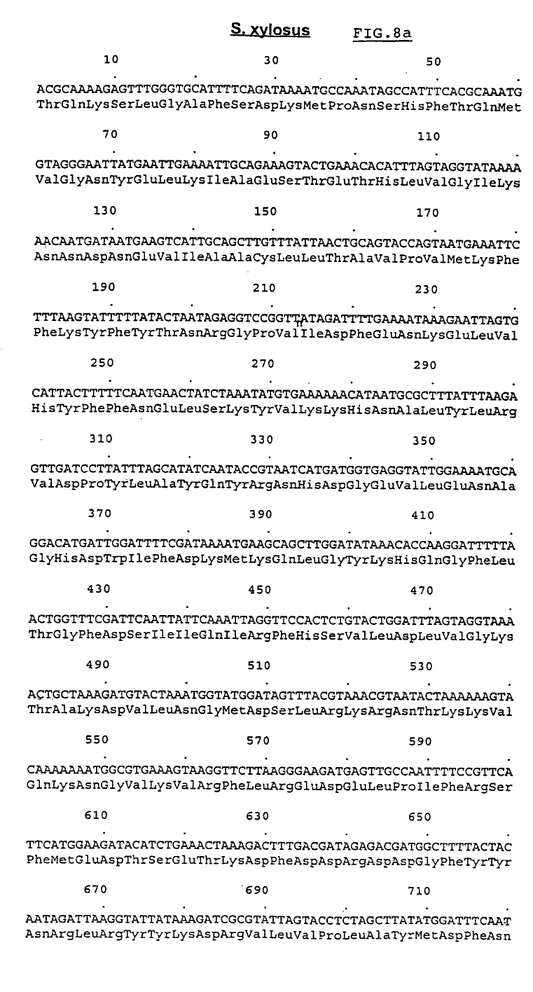

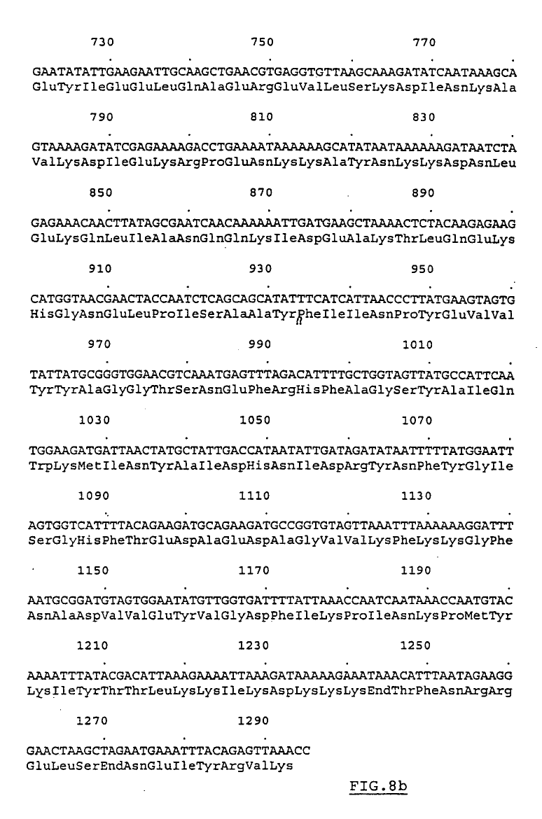

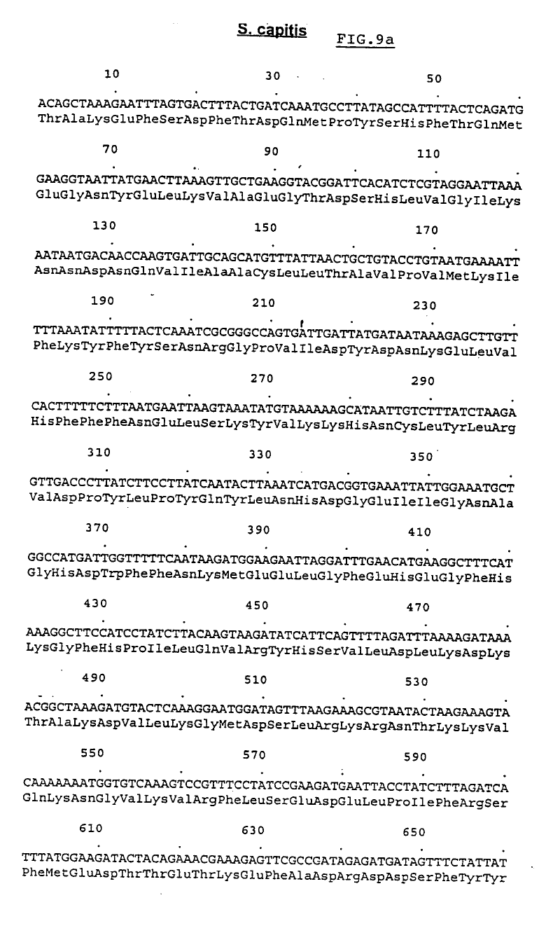

The FIGS. 8a & b represent the complete femA wild type genetic sequence of the strain S. xylosus.

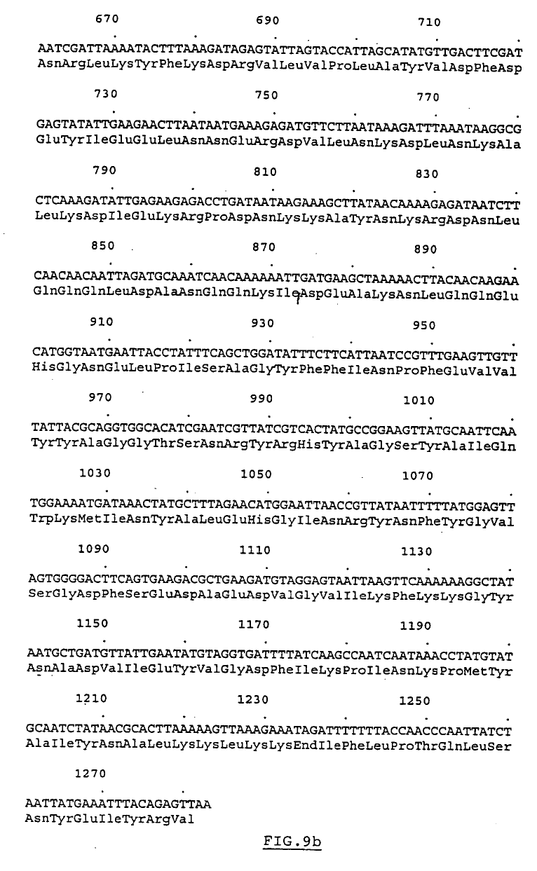

The FIGS. 9a & b represent the complete femA wild type genetic sequence of the strain S. capitas.

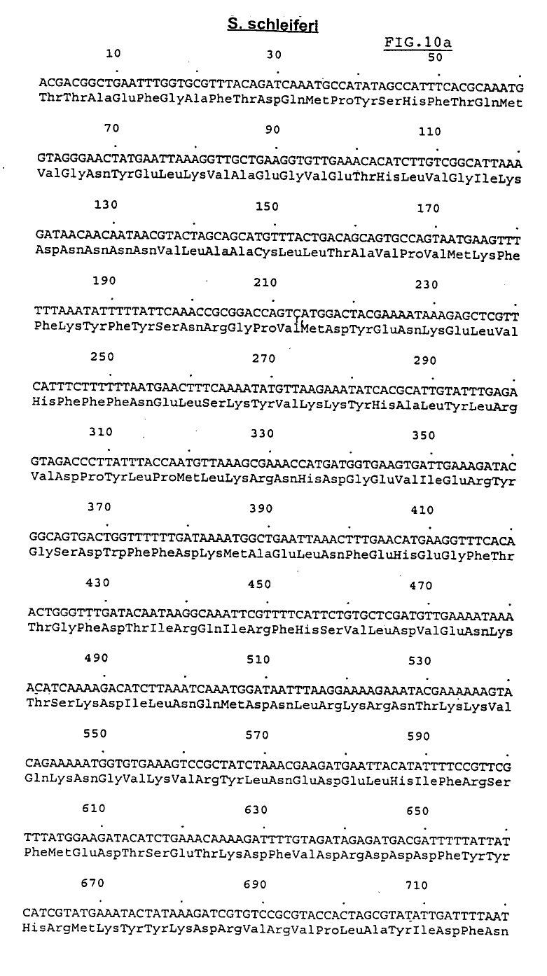

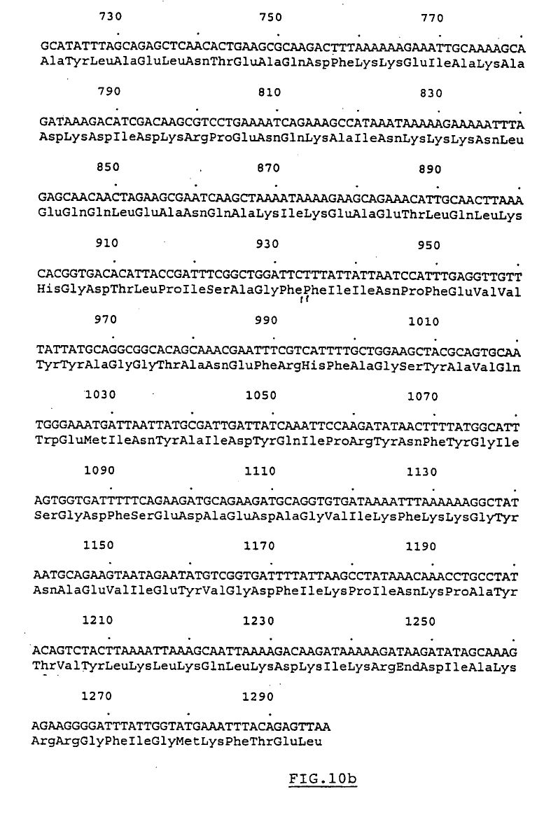

The FIG. 10a & b represent the complete femA wild type genetic sequence of the strain S. schleiferi.

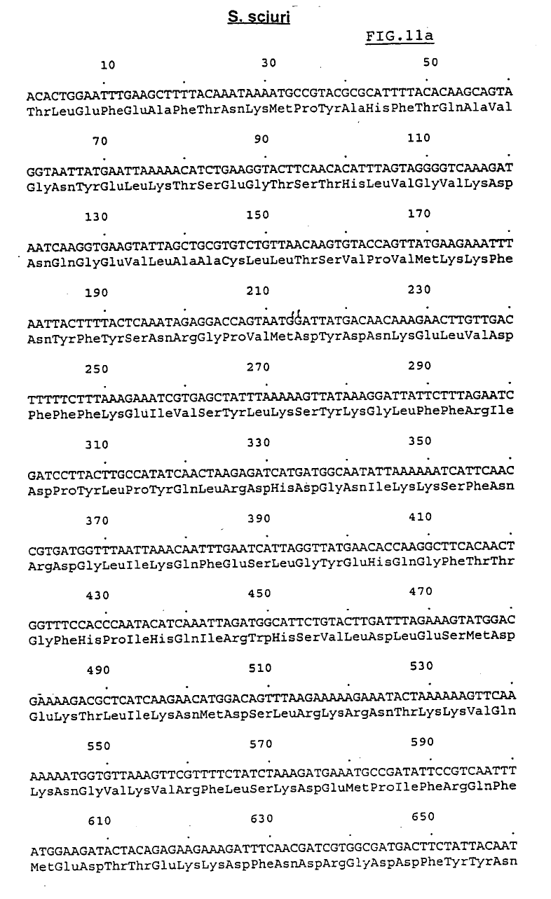

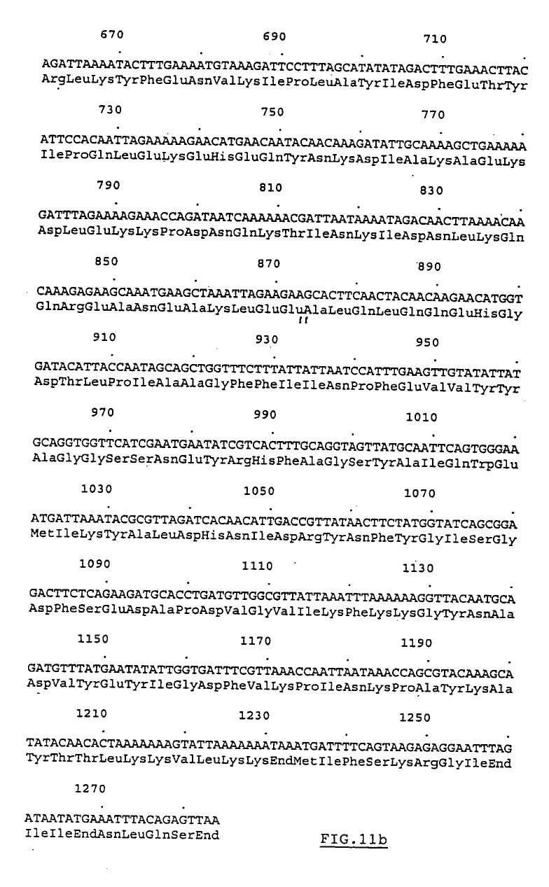

The FIGS. 11a & b represent the complete femA wild type genetic sequence of the strain S. sciuri.

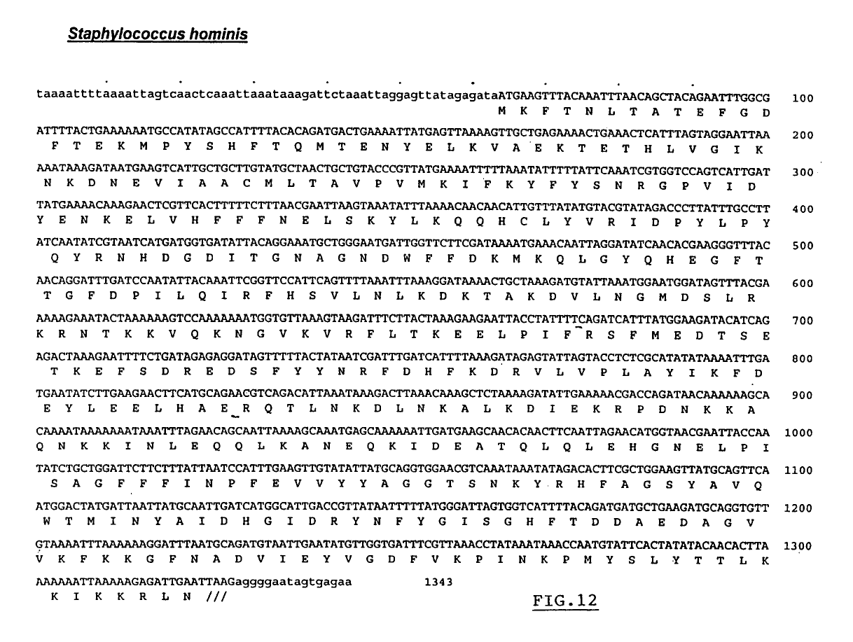

The FIG. 12 represents the complete femA wild type genetic sequence of the strain S. hominis.

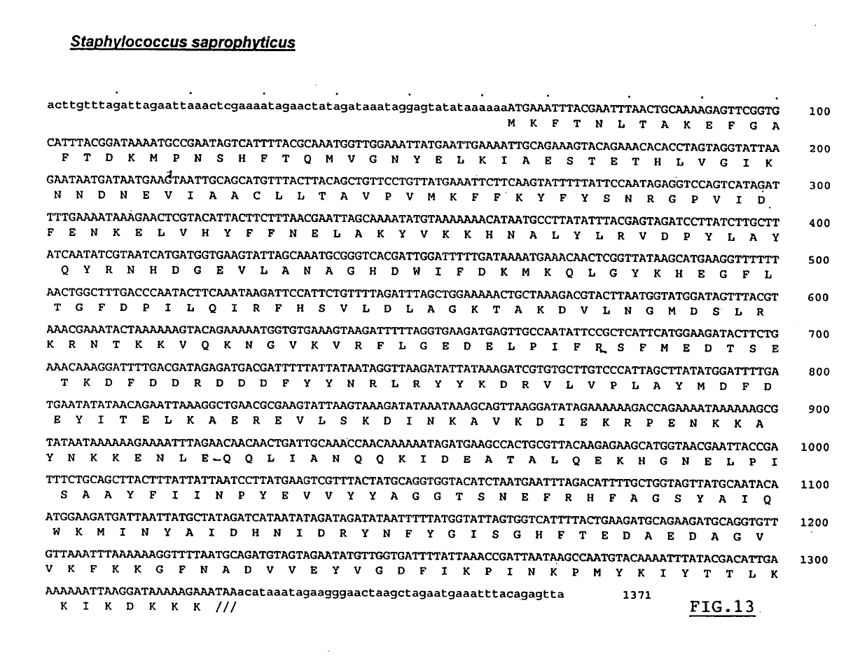

The FIG. 13 represents the complete femA wild type genetic sequence of the strain S. saprophyticus.

### EXAMPLES

### Example 1

### Sequencing Strategy

Fragments of the femA genes from S. hominis and S. saprophyticus have been obtained by PCR amplification, in low stringency annealing conditions. Primers used for amplification are matching the potentially conserved regions and have been designed according to sequences homologies between S. aureus, S. sapropyticus and S. epidermidis femA nucleotide sequences. For both S. hominis and S. saprophyticus species, 5 partially overlapping fragments have been synthesised allowing the sequencing of the entire femA genes (FIG. 1).

### Example 2

### Identification of a Consensus Sequence

Alignment of the nucleotide sequences of femA genes from S. hominis and S. saprophyticus as well as with femA genes sequenced to date from S. aureus (GenBank accession number M23918), S. epidermidis (GenBank accession number U23713) and S. haemolyticus is presented in FIG. 3 and has allowed to propose a “consensus” femA nucleotide sequence (CNS) whose genomic organisation displays highly conserved regions flanked by variable ones. On this basis, interspecies phylogenetic variations could be exploited to design genotyping strategies for species-specific identification of Staphylococci. The “consensus” sequence is therefore a powerful molecular tool for specific diagnostic of staphylococcal infections.

### Example 3

### Sequencing of other Staphylococcal femA Genes

The consensus sequence can be exploited for designing universal primers allowing the production, under permissive annealing conditions, of overlapping PCR products whose sequencing will identify the entire femA sequence.

### Example 4

### Differential Diagnosis between S. aureus. S. epidermidis, S. hominis and S. saprophyticus by Reverse Hybridisation

The Inventors have set up a reverse hybridisation assay for rapid and combined identification of the most clinically relevant Staphylococci species, and their mecA status. Two sets of primers, chosen in a conserved domain of the consensus sequence (bioU1-bioU3 and fem1-fem3bio), amplifying a 286 and bio-220 bp fragments, respectively) were synthesised. Species-specificity of femA amplicons was insured by the genomic variability between the conserved regions. FemA probes were immobilised on nylon strips. Hybridisation was performed with biotinylated femA PCR fragments from the strain of interest. The strategy was first assessed with ATCC strains (S. aureus, S. epidermidis, S. hominis and S. saprophyticus) (FIG. 4). Specificity was identified by standard methods. Accuracy was 100% for species identification.

### Example 5

### Differential Diagnosis between Staphylococcal Species

This assay is able to identify any staphylococcal species if following requirements are fulfilled:

primers fem1, fem2 and fem3bio are universal for Staphylococci;

there is a wide enough phylogenetic variation between any CNS species to promote a specific hybridisation.

The first requirement is fulfilled for, i.e., S. haemolyticus, S. capitis, S. cohnii, S. xylosus, S. simulans, S. lugdunensis, S. schleiferi and S. warneri strains (FIG. 5).

### Example 6

### Multiplex Amplification of femA and mecA Genetic Determinants for a Molecular Diagnosis of a Specific Staphylococcal Infection

A total of 48 patients treated in 4 contiguous intensive cares units were included in the study. Endotracheal aspirates (ETA) were collected from the patients and submitted to the multiplex PCR analysis according to the technique described by Vannuffel et al. (1995). Clinical specimens were homogenised in 5 ml of TE buffer (20 mM TRIS HCl, pH 8.0, 10 mM EDTA) containing 2% (w/v) SDS.

The homogenate (1.5 ml) was then centrifuged for 5 minutes at 7500×g. The cellular pellet was washed once with TE buffer lysed in the presence of 1% (v/v) Triton X-100 and 50 μg of lysostaphin (Sigma) and incubated for 15 minutes at 37° C. Lysis was completed by adding 100 μg of proteinase K (Boehringer). The lysate was incubated for another 5 minutes at 55 ° C. and 5 minutes at 95° C., and centrifuged at 4000×g for 5 minutes.

In order to purify bacterial DNA, 200 μl of supernatant were then filtered on a Macherey-Nagel Nucleospin C+T® column and eluted with 200 μl sterile H2O. Two different amounts of DNA suspension (2 μl and 200 μl) were submitted to multiplex PCR amplification with the primers 5′-TGGCTATCGTGTCACAATCG-3′ (SEQ ID NO: 38)and 5′-CTGGAACTTGTTGAGCAGAG-3′ (SEQ ID NO: 39) for mecA and the above-described primers for femA, yielding different fragments.

femA and mecA signals were found in specimens containing either susceptible S. aureus (n=10) and methycillin-resistant coagulase-negative Staphylococci (n=6) respectively. On the other hand, no signal was obtained from ETA gram-negative bacteria (n=5) as well as MS-CNS (n=6) and from 5 ETA containing normal pharyngeal flora.

This multiplex PCR strategy for detecting Staphylococci in ETA was completed in less than 6 hours either on the day of the samples' collection. This is an advantage with respect to the time required to conventional identification and susceptibility tests (48 to 72 hours).

### Example 7

### Amplification, Cloning and Sequencing of other femA Genes

Two primers were selected among the conserved parts of the consensus sequence for the amplification of the femA gene.

These primers are femS1, femS2 and femAS1 (anti-sense primer). ADN from strains of Staphylococcus hominis, saprophyticus, haemolyticus, lugdunensis, schleiferi, sciuri, xylosus, simulans, capitis, gallinarum, cohnii and wameri were amplified from said primers and amplification fragments were cloned in the vector pCR®-XLTOPO and introduced by electroporation in E. coli cells TOP10 (TOPO XL PCR Cloning Kit®, Invitrogen, Carlsbad, Calif.).

Amplified fragments of strain S. lugdunensis, schleiferi, sciuri, xylosus, and capitis were sequenced by Taq Dye Deoxy Terminator Cycle® sequencing on a ABI 277 DNA sequencer® (PE Applied Biosystems, Foster City, Calif.) by the following primers:

femS1 or femS2 or femAS1

fsq1S and fsq1AS

fsq2S and fsq2AS

fsq3S and fsq3AS

fsq4S and fsq4AS

fsq5S and fsq5AS

fsq6S and fsq6AS

### References

1. Alborn W. E. Jr et al., Gene 180: 177-81 (1996)

2. Berger-Bächi B. et al, Mol Gen Genet 219: 263-9 (1989)

3. Berger-Bächi B. et al., Antimicrob. Agents Chemother. 36: 1367-73 (1992)

4. Chackbart et al., Antimicrobial Agent Chemotherapy 33: 991-999 (1989)

5. de Lancastre H. et al., Antimicrob. Agents Chemother. 38: 2590-8 (1994)

6. Hiramatsu K. et al., FEBS Letters 298: 133-6 (1992)

7. Hurlimann-Dalel R. L. et al., Antimicrob. Agents Chemother. 36: 6+17-21 (1992)

8. Kizaki M. et al., J. Hosp. Infect. 28: 287-95 (1994)

9. Kleeman K. T. et al., J. Clin. Microbiol. 31: 1318-1321 (1993)

10. Refshal K. et al., J. Hosp. Infect. 22(1): 19-31 (1992)

11. Ryffel C. et al., Gene 94: 137-8 (1990)

12. Ryffel C. et al., Antimicrob. Agents Chemother. 38: 724-8 (1994)

13. Rupp M. E. et al., Clin. Infectious Diseases 19: 231-245 (1994)

14. Stranden A. L. et al., J. Bacteriol. 179: 9-16 (1997)

15. Suzuki E. et al., Antimicrob. Agents Chemother. 36: 429-34 (1992)

16. Unal S. et al., J. Clin. Microb. 30: 1685-1691 (1992)

17. Vannuffel P. et al., J. Clin. Microb. 33: 2864-2867 (1995)

18. Vannuffel . et al., J. Clin. Microb. 36: 2366-2368 (1998)

What is claimed is:

1. An isolated and purified nucleotide sequence corresponding to SEQ ID NO:1, homologues or parts thereof.

2. An isolated and purified nucleotide sequence according to claim 1, wherein said homologues comprise SEQ ID NOs: 40, 42, 44, 46, 48, 50, 52 and 54.

3. An isolated and purified nucleotide sequence according to claim 2, wherein said homologues have more than 85% homology to SEQ ID NOs: 40, 42, 44, 46, 48, 50, 52 and 54.

4. An isolated and purified nucleotide sequence according to claim 2, wherein said homologues have more than 90% homology to SEQ ID NOs: 40, 42, 44, 46, 48, 50, 52 and 54.

5. An isolated and purified nucleotide sequence according to claim 1, wherein said parts of SEQ ID NO:1 are nucleotide sequences having between 25 and 350 bp, which have more than 60% homology to SEQ ID NO:1.

6. An isolated and purified nucleotide sequence according to claim 1, wherein said parts of SEQ ID NO:1 are nucleotide sequences having between 25 and 250 bp, which have more than 60% homology to SEQ ID NO:1.

7. An isolated and purified nucleotide sequence according to claim 1, wherein said parts of SEQ ID NO:1 are nucleotide sequences having between 25 and 45 bp, which have more than 60% homology to SEQ ID NO:1.

8. An isolated and purified nucleotide sequence according to claim 1, wherein said parts of SEQ ID NO:1 comprise SEQ ID NOS: 2-28, 56-64.

9. An isolated and purified nucleotide sequence according to claim 1, wherein said parts of SEQ ID NO:1 comprise SEQ ID NOS: 65-83.

10. Use of an isolated and purified nucleotide sequence corresponding to SEQ ID NO:1, homologues or parts thereof for the identification of at least two Staphylococci species.

11. Diagnostic device for the identification of at least two Staphylococci species comprising at least one oligonucleotide which has more than 60% homology to SEQ ID NO:1 and at least one isolated or purified oligonucleotides which has less than 50% homology to SEQ ID NO:1.
